(19) United States
(12) Patent Application Publication
(10) Pub. No.: US 2025/0041546 A1
(43) Pub. Date: Feb. 6, 2025

(54) LUNG REPLICA AND USES THEREOF

(71) Applicants: Catherine Ann Fromen, Wilmington, DE (US); IAN Woodward, Newark, DE (US)

(72) Inventors: Catherine Ann Fromen, Wilmington, DE (US); IAN Woodward, Newark, DE (US)

(73) Assignee: University of Delaware, Newark, DE (US)

(21) Appl. No.: 18/709,078

(22) PCT Filed: Nov. 9, 2022
(86) PCT No.: PCT/US2022/049355

§ 371 Date: May 10, 2024

Related U.S. Application Data
(60) Provisional application No. 63/278,131, filed on Nov. 11, 2021.

(51) Int. Cl. A61M 16/00

(52) U.S. Cl. CPC A61M 16/0006

(57) ABSTRACT

The invention provides a lung replica to approximate spatial deposition in the human airway during breath. The lung replica comprises an upper airway compartment comprising an upper airway space: one or more lobe compartments, comprising two or more generation regions, a compressible terminal unit, and lattices in the two or more generation regions, and each comprising a lobar airway space connected with the upper airway space; and one or more modular components. The one or more modular components actuate the one or more lobe compartments to compress or expand. An exhaled airflow is generated when the one or more lobe compartments are compressed while an inhaled airflow is generated when the one or more lobe compartments are expanded. Also provided is a method for exchanging airflow in the lung replica. Further provided is a method for preparing a lobe compartment or a lung replica.

### CROSS-REFERENCE TO RELATED APPLICATION

This application claims priority to U.S. Provisional Application No. 63/278,131, filed Nov. 11, 2021, and the contents of which are incorporated herein by reference in their entireties for all purposes.

### REFERENCE TO U.S. GOVERNMENT SUPPORT

This invention was made with government support under Grant No. R35GM42866A from the National Institutes of Health. The United States has certain rights in the invention.

### FIELD OF THE INVENTION

This invention relates generally to a lung replica simulating breathing conditions in an analogous lobe or lung and uses thereof for assessing airflow exchange and spatial deposition of aerosols in a full-volume lobe or lung under the simulated breathing conditions.

### BACKGROUND OF THE INVENTION

The localized deposition of inhaled particles and specifically the active pharmaceutical ingredient (API) within the patient lung is the most significant predictor of therapeutic outcomes following inhalation therapy and is an important input to infer both local drug action and systemic drug pharmacokinetics (PK). Despite this fact, predictive physical models of aerosol deposition in the entire respiratory tract are notably absent in the field.

The lung airway is a network of asymmetric bifurcations that varies by sex, age, and disease, which can be grouped in macroscopic lobes: three on the right and two on the left. In the respiratory tract, air flows through the pharynx, the larynx and then trachea, at which point the airway begins to branch through asymmetric bifurcations.⁶ From the trachea (“Generation” 0, G0), the airways divide as many as 23 times, passing through main, lobar, and segmental bronchi (G1-G15), bronchioles (G16-G19), alveolar ducts (G20-22) and the alveoli (G23).

Particulates deposit into the lung during breath, with objects having a mass median aerodynamic diameter (MMAD) between ˜1 μm-5 μm depositing at high efficiency via impaction, diffusion, and/or sedimentation, depending on the particle inertia and the local airflow. Predicting deposition is challenged by the non-uniform airflows, including complex secondary flows, vortices, eddies, and recirculating flows generated from the non-uniform bifurcations, uneven lobe distributions, and lung movement. Thus, local lung structure and breathing pattern control deposition, increasing with slow inhalation and breath holding and varying by total ventilation and posture. While conceptual understanding of how aerosols deposit under varied breathing conditions can be built from clinical observation, accurate spatial measurements and a priori predictions remain scarce, especially across patient populations presenting with varied lung volumes, structures, and breathing capabilities. Current preclinical models of the human airway fail to provide spatial measurements following aerosol deposition under breathing conditions.

### Experimental Measurements

Historically, entire doses of inhaled therapeutics are characterized mainly by their aerodynamic particle size distribution (APSD). The Andersen Cascade Impactor (ACI) and Next Generation Impactor (NGI) predominate as the in vitro sizing standard of inhaled therapies in the pharmaceutical industry due to their inexpensive implementation and established protocols. Aerosols are sized within cascade impactors (CI) after passing through a series of nozzles and collection plates designed to separate particles by inertial impaction. Both ACI and NGI systems are run under constant inhalation flow rates using a vacuum and approximate human deposition based on the distribution of the deposited dose. However, both CIs are known to commonly overestimate the available dosage and fail to accurately capture lung architecture or breath. Attempts to improve in vitro-in vivo correlations (IVIVC) of CIs include use of the Alberta Throat induction port, which incorporates averaged adult upper airway features, and the addition of a breath simulator to recreate inhaler actuation.

Alternative approaches have emerged to overcome the lack of spatial deposition assessment inherent to CIs using physiologically-relevant models that mimic aspects of lung structure. Beginning with either human or animal cadaver, full airway casts and tracheal molds have been generated from a decellularized organ and used to study deposition. Fine airway features especially the alveoli are lost during the decellularization process, limiting these studies to the upper portion of the airspace. Understandably, these top down approaches are costly, fabrication intensive, and require a high degree of expertise that prevents high throughput deposition studies. Recent bottom-up efforts have included use of additive manufacturing or injection molding techniques to generate hollow lung phantoms. These models are made from rigid materials, and similar to airway casts, are unable to reproduce alveolar airway features due to manufacturing limitations. Therefore, these instances primarily consist of mouth-throat replicas and truncated airway trees. Most significantly, these whole-dose approaches typically use inhalation-only flows, despite deposition occurring over multiple breathing cycles. Alternative inventions include breathing simulators; these machines use piston-cylinder or bellows systems to generate representative airflow profiles for clinical education and training, as well as medical device testing and validation. All are exclusively mechanical devices, where the internal components do not provide any lung function or potential for dosage measurement.

### Periodic Porous Media: Lattices

Lattices are one class of additive-only structures which have great potential in directing transport phenomena because they are highly ordered, scalable, and modular. Similar to foams, lattices are high-porosity, strut-based structures that are defined by repeating unit cells. Unlike their stochastic counterparts, lattices can be designed with defined dimensions, porosity, and surface area in an ordered arrangement, enabling a plethora of opportunities for prescribing features of aerosol transport. Porous lattice AM structures contain cylindrical struts which share similarities with fibrous filters and open foams. Lattice structures are comprised of a vast design space but can be often specified with a smaller number of parameters, including unit cell geometry, unit cell size, and element thickness. Defining these features will lead to prescribed porosity, specific surface area, and hydraulic diameter that can facilitate the selection between lattice configurations at the single unit cell level to the overall porous media features. The hydraulic diameter here is especially key; it is a characteristic length used for flows in non-cylindrical shapes and is defined as four times the open area divided by the wetted perimeter.

Single phase fluid flows through porous lattice structures have been described by our group and others. These flows largely follow those of other porous media. Aerosol deposition and filtration mechanisms of lattices derive from those described by flow past a single cylinder, including inertial impaction, diffusion, gravitational settling, interception, and electrostatic interactions. The relative contributions of these mechanisms largely depend on the background flow regime and aerosol particle size.

There remains a need for a preclinical tool for measuring spatial deposition of an orally inhaled and nasal drug product (OINDP) under simulated breathing conditions.

### SUMMARY OF THE INVENTION

The inventors have surprisingly discovered the integration of lattice regions within a whole volume lung replica based on sealed airway lobe units provides a faithful representation of spatial airway deposition in a human lung.

A lung replica is provided. The lung replica comprises (a) an upper airway compartment comprising an upper airway space; (b) one or more lobe compartments, wherein each of the one or more lobe compartments comprises two or more generation regions, a compressible terminal unit, and lattices in the two or more generation regions, and each of the one or more lobe compartments comprises a lobar airway space connected with the upper airway space; and (c) one or more modular components, wherein the one or more modular components actuate the one or more lobe compartments to compress or expand, whereby an exhaled airflow is generated when the one or more lobe compartments are compressed, and an inhaled airflow is generated when the one or more lobe compartments are expanded. The lung replica may comprise 1-18 lobe compartments. Each of the lobe compartments may comprise 2-23 generation regions.

The lung replica may have a total fully expanded lung airway volume of 0.1-7 L. The lung replica may have a total fully compressed lung airway volume of 0.01-3 L.

The upper airway compartment may comprise a wall on the outer surface of the upper airway compartment, and the wall may have a thickness of 0.1-10 mm.

The upper airway compartment may comprise a proximal compartment and a distal compartment, the proximal compartment may comprise a first opening, and the distal compartment may be connected with the one or more lobe compartments. The proximal compartment may further comprise a second opening.

The lung replica may have a total fully expanded lobar airway volume of 0.1-7 L. The lung replica may have a total fully compressed lobar airway volume of 0.01-3 L.

The lung replica may have a total lobe internal surface area of 1-100 m².

Each of the one or more lobe compartments may comprise a shell on the outer surface of each of the one or more lobe compartments, and the shell may have a thickness of 0.1-10 mm. The shell may have a Shore hardness in the range from Shore 00 30 to Shore D 90.

The terminal unit may have a fully expanded alveolar airway volume of 0.1-7 L. The terminal unit may be in the form of a sealed hemisphere, bellows, or frustum.

Each of the two or more generation regions may have a unique property selected from the group consisting of porosity, specific surface area and hydraulic diameter.

The lattices may have a porosity (ε) of 0.1-1. The lattices may have a dimensionless specific surface area (aᵥ·l_{c}) of 0.1-10. The lattices may have a hydraulic diameter (d_{H}) of 0.1-15 mm. The lattices may have a Shore hardness in the range from Shore 00 30 to Shore D 90. The lattices may comprise polymeric periodic porous media compartments. The lattices may comprise unit cells. The unit cells may be asymmetric. The unit cells may be non-uniform and patterned.

Each of the one or more modular components may be at an end of at least one of the one or more lobe compartments. The modular component has a linear rate of translated motion at 0.1-10 mm/s.

For each lung replica of the present invention, a method for exchanging airflow in the lung replica is provided. The method comprises (a) compressing the one or more lobe compartments, whereby an exhaled airflow is generated; and (b) expanding the one or more lobe compartments, whereby an inhaled airflow is generated. As a result, the lung replica exchanges airflow. Step (a) may be before step (b). Step (a) may be after step (b).

The airflow exchange method may further comprise holding the inhaled airflow in the lung replica for at least 1 second between step (b) and step (a). The one or more lobe compartments may be fully compressed in step (a). The one or more lobe compartments may be fully expanded in step (b).

The airflow exchange method may further comprise quantifying the exhaled airflow.

The airflow exchange method may further comprise quantifying the inhaled airflow.

The airflow exchange method may further comprise repeating steps (a) and (b).

According to the airflow exchange method, the inhaled airflow may comprise an aerosol, and the aerosol may be deposited in the lung replica. The airflow exchange method may further comprise determining the deposition profile of the aerosol in the lung replica. The airflow exchange method may further comprise modifying the deposition profile. The airflow exchange method may further comprise modifying at least one of the two or more generation regions, the lattices, the flow rate of the inhaled airflow, the amount of the inhaled airflow, the flow rate of the exhaled airflow, the amount of the exhaled airflow, the duration of step (a), and/or the duration of step (b).

A method for preparing a lobe compartment is provided. The lobe compartment comprises a lobe airway space and a generation region based on an analogous airway generation in an analogous lobe. The lobe compartment preparation method comprises: (a) forming the lobar airway space in the lobe compartment with a series of connected circles having gradually changing diameters along the length of the lobe compartment; (b) adding a lattice into a circle in the connected circles to form a generation region, wherein the generation region has a cross sectional area based on a cumulative cross sectional area of the analogous airway generation, scaled in inverse proportion to porosity of the lattice, and a depth based on a length of the analogous airway generation; and (c) forming a terminal unit in the lobe compartment, whereby the lobe compartment is prepared. The lobe compartment preparation method may further comprise repeating step (b) for one or more times. The prepared lobe compartment comprises two or more generations based on all of the analogous airway generations in the analogous lobe. The volume of the lobar airway space in the prepared lobe compartment may be equal to 80-120% of the airway volume in the analogous lobe.

For each lung replica of the present invention, a preparation method is prepared. The lung replica preparation method comprises (a) providing an upper airway compartment, wherein the upper airway compartment comprises an upper airway space; (b) providing one or more lobe compartments prepared according the method of the present invention; (c) connecting the upper airway space with the lobar airway space in each of the one or more lobe compartments; and (d) providing one or more modular components, wherein the one or more modular components actuate the one or more lobe compartments to compress or expand, whereby an exhaled airflow is generated when the one or more lobe compartments are compressed, and an inhaled airflow is generated when the one or more lobe compartments are expanded.

### BRIEF DESCRIPTION OF THE DRAWINGS

FIG. 1 is a diagram of whole volume lung replica design and features. A indicates an extrathoracic airway compartment (mouth only shown but nasal passage may also be included). B indicates a tracheobronchial airway compartment. C indicates a detachable and sealed lobe compartment, wherein the lobe compartment comprises a compressible terminal unit. A cross section of the lobe compartment is shown to the right, where internal lattice elements (D) are indicated. D indicates removable lattice inserts within the lobe compartment. E indicates a component which mechanically actuates the deformable unit.

FIG. 2 shows realization of whole volume lung replica design and features indicated. A indicates an extrathoracic airway compartment (mouth only shown but nasal passage may also be included). B indicates a tracheobronchial airway compartment. A and B are derived from an idealized airway model. C indicates a detachable and sealed lobe compartment, wherein the lobe compartment comprises a compressible terminal unit. The prototype on the left represents dimensions of a full volume airway that is open to demonstrate key components; the prototype to the right (top) represents a scaled small volume lobe that is fully sealed.

FIGS. 3A-E show centerline-based truncation from airway tree. (A) Trachea and bronchi subdivisions are indicated, along with three representative lobar truncation planes (black lines). (B) Three exemplary points of truncation along the main bronchi, showing the point of truncation and the plane of truncation. The plane normal follows the direction of the centerline at the point of truncation. (C) A truncated airway tree where truncation occurs at the main bronchi. (D) A truncated airway tree representing where truncation occurs at the right upper lobar bronchus. (E) A truncated airway tree where truncation occurs at the right upper segmental bronchi (apical, posterior, and anterior).

FIGS. 4A-D show generation of a lobe unit from airway data. (A) Either patient data or morphometric data are used to determine the total cross sectional area and average length for each generation within the lobe. (B) This then defines the cross sectional area and length of the lobe unit horn structure at each generation, as visualized by the concentric rings. (C) The horn structure is then connected by smoothing the generation regions and connecting them with an outer wall for a physical manifestation. After the final combined generation, all remaining airways before the alveoli are represented by extruding the circle of the final generation out to an equivalent volume that is sealed. (D) Lattice designs then populate these internal regions after scaling the cross sectional area for each generation to mimic the local transport properties (i.e. hydraulic diameter, equivalent velocity). An exemplary full volume internal shell in C shows a two-lattice approximation (color coded) with terminal alveolar region.

FIG. 5 shows lattices. (Top) Simplified lifecycle of lattice parts from design for additive manufacturing to application. (Bottom) Photographs of printed parts, adjusted for visual clarity showing large scale open lattice monoliths and representative range of available lattice unit cells demonstrating a representative range of available periodic porous media designs. Scale bars 1 cm.

FIGS. 6A-D show directional lattice designs, including a bidirectional uniform lattice (A and B) and an alternating pyramid lattice (C and D). A) A single unit cell with directionality is populated in B) a full lattice configuration with this pattern such that, as airflow enters from the top to bottom of the cell, it is faced with a more open cross section and less resistance, while the transverse flow results in a smaller cross section and more resistance. C) A top-down and D) side view of a patterned pyramid lattice and full patterned configuration such that a pyramid unit cell is used to impose directionality while the overall part experiences patterning to further tune the flow properties.

FIG. 7 shows an aerosol deposition as a function of a local lattice architecture, with increased unit cell density driving increased spatial deposition.

FIG. 8 shows a test lobe assembly used for deposition trial. (A) Lobe shell encasing lattice structures and mounting to the airway replica. (B) Alveolar shell mounted to the lobe shell, enclosing the lobe and alveolar volume. (C) Proximal lattice unit. (D) Distal lattice unit. (E) Mounting point for modular component. Not shown: nebulizer (delivery device), adapter, and exhaust collection chamber.

FIGS. 9A-C show characteristic geometric properties of exemplary lattice structures, represented in dimensionless form, including A) porosity, B), specific surface area, and C) hydraulic diameter, as a function of dimensionless radius for a range of unit cell geometries. Terms include cell length (l_{c}), strut radius (r), dimensionless radius (r*=r/l_{c}), porosity (ε), specific surface area (aᵥ), and hydraulic diameter (d_{H}).

FIG. 10 are repeatable breathing profiles of a lung replica prototype demonstrating sequential exhalation and inhalation maneuver on the sealed prototype replica.

FIGS. 11A-B shows a lung replica model controlling total volume exchanged at constant flow rate. A) The flow rate (SLPM) vs time (min) is measured at the mouth of the sealed 5-lobe prototype (FIG. 2, top right, same as FIG. 10). B) The same data is plotted as a function of flow rate (SLPM) vs total volume exchanged (L), obtained by integrating the area under the curve from the plot on the left. The series shown (200, 250, 300) indicate the number of steps taken by the motor, corresponding to increasing volumes of air exchanged.

FIG. 12 show a lung replica model controlling peak inlet flow rate at constant volume. A) The flow rate (SLPM) vs time (min) is measured at the mouth of the sealed 5-lobe prototype (FIG. 2 top right, same as FIG. 9). B) The same data is plotted as a function of flow rate (SLPM) vs total volume exchanged (L), obtained by integrating the area under the curve from the plot on the left. The series shown (fast, mid, slow) indicate the rate of steps taken by the motor, corresponding to increasing speeds to generate varied peak flow rates.

FIG. 13 shows breath holds of varied during in a lung replica model. The plot on the left demonstrates the flow rate (SLPM) vs time (min) as measured at the mouth of the sealed 5-lobe prototype (FIG. 2 top right, same as FIG. 10). The plot on the right shows the same data plotted as a function of flow rate (SLPM) vs total volume exchanged (L), obtained by integrating the area under the curve from the plot on the left. The series shown (1 s, 2.5 s) indicate the time between breathing maneuvers, corresponding to the duration of breath hold generated by the replica.

FIG. 14 shows qualitative description of a resultant deposition maps. Under an imposed set of conditions (model geometry, breathing maneuver), regional deposition will be quantified within each of the 5 lobes at every lattice generational position, as well as the amount exhaled. From this quantification, the deposited and exhaled fraction may be determined from the total aerosol dosed, as well as the percent deposited within each local generational region. Darker shared areas represent regions of higher deposition amounts.

### DETAILED DESCRIPTION OF THE INVENTION

The present invention provides a lung replica having an upper airway compartment, a lobe compartment with generation regions, a compressible terminal unit and lattices in the generation regions, and a modular component for generating an exhalation airflow out of the lung replica by compressing the lobe compartment or an inhalation airflow into the lung replica by expanding the lobe compartment. The lung replica may be used for simulating breathing conditions, and assessing spatial deposition of an aerosol in the lung replica under the simulated breathing conditions when the aerosol is included in the inhalation airflow. The invention is based on the inventors' surprising discovery that a physical whole lung replica using lattice regions enables experimental measurement of spatial aerosol deposition and inhaled/exhaled fraction of an emitted dose. The inventors have adopted a complete pipeline approach for creating a whole lung deposition replica model from patient or population lung data with modular components for plug-and-play customization that mimics physiological airway features (i.e., breathing, lobe compartments, aerosol filtration efficiencies). Essential to the lung replica is periodic porous media, i.e., lattices.

The lung replica overcomes limitations of exclusively mechanical breathing simulator devices, where the internal components do not provide any lung function or potential for dosage measurement, by including modular lobe-level units of the entire lung volume with both inhalation and exhalation maneuvers. It is innovative over the prevailing paradigm of stationary in vitro inhalation models, which remain insufficient to correlate to clinical response, to bridge the persistently large gap between preclinical predictions and clinical findings. The lung replica bridges the gap between in vitro and in vivo assessments, providing spatial lobe and generation-specific resolution under breathing profiles through meaningful approximations.

The inventors have developed key inventive concepts in three areas. First, individually actuated lobe-units connected to a patient-derived upper airway geometry to provide lobe-level breathing profiles following mechanical actuation of the unit(s). Second, lobe-units are populated with removable defined periodic-repeating porous media (i.e. lattices) that allow for repeatable and customizable configurations across needed length scales. Third, lattice unit cell geometries collect differential aerosol sizes with varied filtration efficiencies to match physiological airway profiles in health and disease states. To improve the prediction of inhaled medicine efficacy through high-throughput aerosol delivery predictions, knowing where aerosols deposit is the number one predictor of therapeutic efficacy, yet no current approach can provide this level of information. The inventors have unexpectedly discovered a lung replica providing spatial deposition of an entire lung volume under physiological breathing conditions for improved modelling of aerosol deposition within the human airspace.

The inventors have created a physical whole lung replica to enable experimental measurement of spatial aerosol deposition and inhaled/exhaled fraction of emitted aerosol dose. The replica captures the total lung airspace volume that ranges from newborn to adult, for example, from about 0.5 L to about 8 L. This whole lung deposition replica has been realized experimentally based on lung information. Here, “lung info” may include any range of existing models of lung morphometry, breathing pattern, lung volume distribution, and airway resistance/compliance, including measured or interpreted patient-specific metrics. It is readily adaptable to model healthy and diseased airways by leveraging modular components (e.g., lobe, lattices and upper airways) for plug-and-play customization and improved patient predictions. It may accommodate existing mannequins and anatomical phantoms as necessary, integrating all levels of existing lung information.

Breathing maneuvers in the whole volume lung replica model represent a key component. Movement in sealed lobe units creates physiological breathing profiles at the lobe-level. Breathing profiles may be imposed and include sine, square, inspiration, expiration, tidal, and diseased waveforms. Breathing motion may be achieved by deformation of sealed lobe units generated by individually controlled motors. Here, motors or actuators of any type may be used, and either applied tension or compression may create an internal air volume change. Lobe units are of elastic material to be deformable. The material may be 3D printed or cast and the main requirement is that the material is able to enable tension and compression for creating a volume change. Spatial supports may be added to further regulate directionality of deformation. Breathing waveforms may be tuned to match profiles from literature with standard waveforms. Alternative measurements from patient values (spirometry) may be programmed in. The overall flow volumes may be tuned to include tidal volumes within a range of resting to exercise that range of 0-150 LPM. Uniquely, the replica model may impose breath holds through stationary motor holds at controlled positions and time within a breathing maneuver. Overall breathing may be measured at the mouth and within the lobe units. Breathing profiles that mimic features of disease include off-set lobe actuation to mimic altered disease flow profiles, decrease/increase contributions from individual lobes, diseased waveforms (patient-specific, informed by literature).

The inventors have created a lobe unit, also known as lobe compartment, representing an innovative process using lattices. The innovative process may include aspects of disease modeling into the lobe unit.

Lattices allow for spatial deposition measurements within each lobe unit by introducing removable, tunable lattices, which may be periodic repeating porous media. The periodic porous media allow for repeatable and customizable configurations across needed length scales. Lattice unit cell geometries collect differential aerosol sizes with varied filtration efficiencies. Lattices may be highly tunable to match other desired physiological benchmarks such as surface area, flow profiles. Lattices may be removable for convenience and reusability.

Lattice design regions within a lobe unit may be grouped together based on available deposition validation data to match filtration efficiency of human airway. Lattice unit cells tuned by unit cell geometry (e.g., cubic, Kelvin cell or Weaire Phalen), unit cell volume (e.g., length, width, depth), and beam dimensions. Furthermore, the overall pattern of the periodic lattice may include continuous uniform periodic structures, patterning of unit cells and/or unit cell sizes, gradient or discontinuous lattices, obstructions, flow directional patterning (through obstructions or incorporated conditioners, valves), conformation, meshing, or discretization approach that yields programmed orientation or directionality. Lattice designs may be selected to match hydraulic diameter of a corresponding region of a lung. Alternatively, lattice designs may be selected to match the filtration efficiency for designed lattice to human deposition data.

Within a given lung range, one lattice configuration may accommodate range of flow rates, breathing patterns, and/or aerosol sizes. Selection of a lattice may be optimized for the ratio of deposition based on particle size & flow rate (e.g., impaction parameter). Lattices have overlapping geometric properties may be used to accommodate optimization towards this goal.

Alternatively, if single configuration is unable to achieve reasonable validation benchmarks across range of flow rates (Q), breathing patterns and/or aerosol sizes, constraints on model usage may be established for each lattice conformation, resulting in multiple models to cover an experimental or physiological range, for example, Model A comprised of lattice set A using Q 0-20 LPM while Model B comprised of lattice set B using Q 20-40 LPM.

The inventors have discovered that lattice configurations may enable the lung replica to model a disease. From either patient deposition data or interpreting of structural CT data, lattice configurations may be tuned to match an estimated hydraulic diameter and/or filtration efficiency. This allows for a healthy lung replica to be adapted to incorporate new and/or advancing disease features. The model may also use known characterization of airway diseases in the absence of progressive individual models (e.g., COPD GOLD classifications) where data exists. Lattices may then be altered to mimic disease conditions. Total volume, lattice beam thicknesses may be patterned into region-specific spatial disease modifications. Thickness may be applied to surfaces or beams to match airway resistance and compliance corresponding to state of health/disease and progression. Obstructions may be incorporated within lattices or overall change to lattice region. Any surface included in a lattice may be removed/decreased to model airway reconstructions that result in loss of structure as may occur in disease.

An upper airway model from CT scan may be used to create the lung replica. Using either patient-specific or idealized upper airways, lung replica model allows for patient-specific airflows which are critical in downstream deposition. These may be made from either static and rigid parts or from elastic materials that allows for incorporation of deformation. Elastic parts allow for synchronized actuation in glottis region to align with breathing profile, or model an upper airway constriction. The deformation timescale may be tuned by a variety of external stimuli.

The lung replica enables measurement of spatial deposition. Aerosols collected on lattices during breathing maneuvers may be aggregated to generate 3D spatial deposition maps that are a function of lobe unit & generation/lattice position. The replica may accommodate a range of analytical approaches for quantification, including bulk chemical assessment through 1) an analysis of rinsed parts on the removable lattices using appropriate analytical techniques (i.e., inductively coupled-mass spectrometry [ICP-MS], high-performance liquid chromatography [HPLC], MS, or fluorescence), 2) direct imaging (e.g., MRI, CT, Xray, SPECT and confocal), or 3) quartz crystal microbalance (QCM) measurements. The optimal analytical method may depend on the nature of the aerosol and its total dosage administered. The exhaled fraction may be measured by collecting aerosols not deposited on the lung replica and quantified with the same methods as above. This may be done by inserting a chamber over the mouth during the exhalation maneuver. This leads to a new way of characterizing aerosol deposition data, with higher spatial resolution information.

The terms “lung replica,” “lung model” and “lung replica model” are used herein interchangeably and refer to a system or model made of a plurality of components representing essential structures of a lung, including an extrathoracic airway, a tracheobronchial airway and air spaces in lobes.

The lung replica may be a whole-volume lung replica having a total lung airway volume equivalent to that of an adult human lung.

The lung replica may be a small-volume lung replica having a total lobar airway volume smaller than that of a whole volume lung replica. The small-volume lung replica may have about 1-99% of the total lobar airway volume of a whole-volume lung replica. The total lung airway volume of the small-volume lung replica may be in the range from 0.1 L to 7 L. The total lung airway volume of the small-volume lung replica may be equivalent to that of a pediatric lung.

The term “lobar airway volume” as used herein refers to the volume of the lobar airway space in a lobe compartment.

The term “fully expanded lobar airway volume” as used herein refers to the lobar airway volume of a lobe compartment when the lobe compartment is fully expanded.

The term “fully compressed lobar airway volume” as used herein refers to the residual lobar airway volume of a lobe compartment when the lobe compartment is fully compressed.

The term “total lobar airway volume” as used herein refers to the total volume of the lobar airway space in each lobe compartment in a lung replica.

The term “total fully expanded lobar airway volume” as used herein refers to the total lobar airway volume of a lung replica when each lobe compartment in the replica is fully expanded.

The term “total fully compressed lobar airway volume” as used herein refers to the total lobar airway volume of a lung replica when each lobe compartment in the replica is fully compressed.

The term “total lung airway volume” as used herein refers to the combined volume of the total lobar airway volume and the volume of the upper airway space in a lung replica.

The term “total fully expanded lung airway volume” as used herein refers to the total lung airway volume of a lung replica when each lobe compartment in the lung replica is fully expanded.

The term “total lung fully compressed lung airway volume” as used herein refers to the total lung airway volume of a lung replica when each lobe compartment in the lung replica is fully compressed.

The terms “alveolar airway volume” and “alveolar volume” are used interchangeably and refer to the volume of the lobar airway space in the terminal unit in a lobe compartment.

The term “fully expanded alveolar airway volume” as used herein refers to the alveolar airway volume of a lobe compartment when the lobe compartment is fully expanded.

The term “fully compressed alveolar airway volume” as used herein refers to the alveolar airway volume of a lobe compartment when the lobe compartment is fully compressed.

The term “total lobe internal surface area” as used herein refers to the total internal surface area of each lobe compartment in a lung replica. The lattices may be deformable, and the total internal surface area may change when the lobe compartment expands or compresses.

The term “porosity (ε)” as used herein refers to as the ratio of the volume of the voids or open pore space divided by the total volume of the reference space.

The term “specific surface area (aᵥ)” used herein refers to the ratio of the surface area of a solid structure divided by the total volume of the reference space.

The term “dimensionless specific surface area (aᵥ·l_{c})” used herein refers to a nondimensional quantity that allows for rapid comparison for the internal surface area of a given lattice structure and is defined as the product of specific surface area (which has units equal to square mm per cubic mm) and unit cell length (which has units of mm).

The term “hydraulic diameter (d_{H})” as used herein refers to a characteristic length used for flows in non-cylindrical shapes and is defined as four times the open area divided by the wetted perimeter or four times the porosity divided by the specific surface area.

The term “dimensionless radius (r*)” as used herein refers to the ratio of the strut element thickness divided by the cell length.

The term “Shore hardness” used herein refers to a measure of the resistance a material has to indentation and is measured on Shore scales (commonly 00, A, D), where increasing large numbers represent increasingly hard materials and the appropriate corresponding scales include Shore 00, Shore A, and Shore D that measure increasing hardness.

The term “periodic porous media” used herein refers to a metamaterial with regular, well-defined structure that contains open pores or spaces between solid material through which fluid can pass.

The term “unit cell” used herein refers to the smallest portion of a lattice structure that forms the base repeating geometry that defines the periodic porous media. Unit cells are symmetric where the unit cells are comprised of a base geometry that imposes the same flow properties regardless of the unit cell orientation relative to the flow axes. Unit cells are asymmetric where the unit cells are comprised of a base geometry that imposes the different flow properties dependent on the unit cell orientation relative to the flow axes. The unit cells may be non-uniform where the unit cells are designed or manufactured with gradient feature sizes, such as element thickness or cell length. The unit cells may be patterned where multiple unit cells are configured within a lattice.

The term “linear rate of translated motion” used herein refers to the rate of unidirectional motion (compression, expansion) translated to the lobe compartment by an actuator.

The term “exhaled airflow” used herein refers to an airflow moving out of a lung replica when each lobe compartment in the lung replica expands.

The term “inhaled air” used herein refers to an airflow moving into a lung replica when each lobe compartment in the lung replica compresses.

The term “deposition profile” used herein refers to the three-dimensional configuration of aerosol particles which have collected on the defined solid surfaces of the lung replica through mechanisms of inertial impaction, sedimentation, diffusion, interception, and/or electrostatic effects.

The term “analogous lobe” used herein refers to a native lobe in a human individual or computerized lobe based on lobes of a human population, the information of which native or computerized lobe is used to generate a lobe compartment.

The term “analogous lung” used herein refers to a native lung in a human individual or a computerized lung based on lungs of a human population, and the information of which native lung or computerized lung is used to generate a lung replica.

The present invention provides a lung replica. The lung replica comprises an upper airway compartment, one or more lobe compartments, and one or more modular components. The upper airway compartment comprises an upper airway space. Each lobe compartment comprises at least two generation regions, a compressible terminal unit, and lattices in the generation regions. Each lobe compartment comprises a lobar airway space connected with the upper airway space at the proximal end of the lobe compartment. Each modular component actuates one or more lobe compartments to compress or expand such that an airflow is generated. For example, each lobe compartment is actuated by one modular component. An exhaled airflow is generated when the one or more lobe compartments are compressed. An inhaled airflow is generated when the one or more lobe compartments are expanded.

The lung replica may have a total lung airway volume equivalent to that of a whole volume lung. The volume of the upper airway space may be equivalent to the combined volume of the extrathoracic airway and tracheobronchial airway in the lung. The volume of a lobar airway space in a lobe compartment may be equivalent the volume of the airway in a lobe. The total volume of each lobar airway space in the lung replica may be equivalent to the total volume of the airway in each lobe in the lung. The volume of the lobar airway space in a terminal unit in a lobe compartment may be equivalent to the alveolar volume of a lobe.

The lung replica may have a total lung airway volume equivalent to the total airway volume of a lung. The volume of the upper airway space may be equivalent to the combined volume of the extrathoracic airway and tracheobronchial airway of a lung. The lobar airway volume of a lobe compartment may be equivalent the airway volume of a lobe. The total lobar airway volume of a lung replica may be equivalent to the total lobar airway volume of a lung. The alveolar airway volume of a lobe compartment may be equivalent to the alveolar volume of a lobe.

The lung replica may have a total fully expanded lung airway volume of about 0.1-10 L, 0.1-9 L, 0.1-8 L, 0.1-7 L, 0.1-6 L, 0.1-5 L, 0.1-4 L, 0.1-3 L, 0.1-2 L, 0.1-1 L, 0.1-0.5 L, 1-10 L, 1-9 L, 1-8 L, 1-7 L, 1-6 L, 1-5 L, 1-4 L, 1-3 L, 5-2 L, 5-10 L, 5-9 L, 5- 8 L, 5-7 L or 5-6 L. The total fully expanded lung airway volume may be about 0.1-7 L.

The lung replica may have a total fully compressed lung airway volume of about 0.01-5 L, 0.01-4 L, 0.01-3 L, 0.01-2 L, 0.01-1 L, 0.01-0.5 L, 0.01-0.1 L, 0.05-4 L, 0.5-4 L, 0.5-3 L, 0.5-2 L, 0.5-1 L, 1-5 L, 1-4 L, 1-3 L or 1-2 L. The total fully compressed lung airway volume may be about 0.01-3 L.

The upper airway compartment may comprise a wall on the outer surface of the upper airway space. The wall may have a thickness of about 0.1-10 mm, 0.1-9 mm, 0.1-8 mm, 0.1-7 mm, 0.1-6 mm, 0.1-5 mm, 0.1-4 mm, 0.1-3 mm, 0.1-2 mm, 0.1-1 mm, 0.1-0.5 mm, 0.5-10 mm, 0.5-9 mm, 0.5-8 mm, 0.5-7 mm, 0.5-6 mm, 0.5-5 mm, 0.5-4 mm, 0.5-3 mm, 0.5-2 mm, 0.5-1 mm, 1-10 mm, 1-9 mm, 1-8 mm, 1-7 mm, 1-6 mm, 1-5 mm, 1-4 mm, 1-3 mm, 1-2 mm, 2-10 mm, 2-9 mm, 2-8 mm, 2-7 mm, 2-6 mm, 2-5 mm, 2-4 mm, 2-3 mm, 3-10 mm, 3-9 mm, 3-8 mm, 3-7 mm, 3-6 mm, 3-5 mm, 3-4 mm, 4-10 mm, 4-9 mm, 4-8 mm, 4-7 mm, 4-6 mm, 4-5 mm, 5-10 mm, 5-9 mm, 5-8 mm, 5-7 mm or 5-6 mm. The wall may have a thickness of about 2-4 mm, for example, 3 mm.

The upper airway compartment may comprise a proximal compartment and a distal compartment. The proximal compartment may have an opening. The opening may represent a connection between a lung and a mouth. The airflow may move into or out of the lung replica through the opening. The proximal compartment may further comprise an additional opening. The additional opening may represent a connection between a lung and a nasal passage. The distal compartment may be connected with each lobe compartment at the proximal end of the lobe compartment.

The lung replica may comprise 1-18 lobe compartments. For example, the lung replica comprises 5 lobe compartments, corresponding five lobes in a human lung. Where there are two or more lobe compartments, the modular components may actuate the lobe compartments in coordinated movements of compression or expansion. The coordinated movements may be synchronized movements when the lobe compartments move in the same way to the same extent for the same duration and frequency.

The lung replica may have a total fully expanded lobar airway volume of about 0.1-10 L, 0.1-9 L, 0.1-8 L, 0.1-7 L, 0.1-6 L, 0.1-5 L, 0.1-4 L, 0.1-3 L, 0.1-2 L, 0.1-1 L, 0.1-0.5 L, 1-10 L, 1-9 L, 1-8 L, 1-7 L, 1-6 L, 1-5 L, 1-4 L, 1-3 L, 5-2 L, 5-10 L, 5-9 L, 5- 8 L, 5-7 L or 5-6 L. The lung replica may have a total lobe fully expanded airway volume of about 0.1-7 L.

The lung replica may have a total fully compressed lobar airway volume of about 0.01-5 L, 0.01-4 L, 0.01-3 L, 0.01-2 L, 0.01-1 L, 0.01-0.5 L, 0.01-0.1 L, 0.05-4 L, 0.5-4 L, 0.5-3 L, 0.5-2 L, 0.5-1 L, 1-5 L, 1-4 L, 1-3 L or 1-2 L. The lung replica may have a total lobe fully compressed airway volume of about 0.01-3 L.

The lung replica may have a total lobe internal surface area of about 0.1-1,000 m², 0.1-500 m², 0.1-100 m², 0.1-50 m², 0.1-10 m², 0.1-5 m², 0.1-1 m², 1-1,000 m², 1-500 m², 1-100 m², 1-50 m², 1-10 m², 10-1,000 m², 10-500 m², 10-100 m² or 10-50 m². The lung replica may have a total lobe internal surface area of about 1-100 m².

Each lobe compartment may comprise a shell on the outer surface of the lobe compartment. The shell may have a thickness of about 0.1-10 mm, 0.1-9 mm, 0.1-8 mm, 0.1-7 mm, 0.1-6 mm, 0.1-5 mm, 0.1-4 mm, 0.1-3 mm, 0.1-2 mm, 0.1-1 mm, 0.1-0.5 mm, 0.5-10 mm, 0.5-9 mm, 0.5-8 mm, 0.5-7 mm, 0.5-6 mm, 0.5-5 mm, 0.5-4 mm, 0.5-3 mm, 0.5-2 mm, 0.5-1 mm, 1-10 mm, 1-9 mm, 1-8 mm, 1-7 mm, 1-6 mm, 1-5 mm, 1-4 mm, 1-3 mm, 1-2 mm, 2-10 mm, 2-9 mm, 2-8 mm, 2-7 mm, 2-6 mm, 2-5 mm, 2-4 mm, 2-3 mm, 3-10 mm, 3-9 mm, 3-8 mm, 3-7 mm, 3-6 mm, 3-5 mm, 3-4 mm, 4-10 mm, 4-9 mm, 4-8 mm, 4-7 mm, 4-6 mm, 4-5 mm, 5-10 mm, 5-9 mm, 5-8 mm, 5-7 mm or 5-6 mm. The shell may have a thickness of about 2-4 mm, for example, 3 mm. The shell may have a Shore hardness in the range from Shore 00 30 to Shore D 90.

The terminal unit may have a fully expanded alveolar airway volume of about 0.1-10 L, 0.1-9 L, 0.1-8 L, 0.1-7 L, 0.1-6 L, 0.1-5 L, 0.1-4 L, 0.1-3 L, 0.1-2 L, 0.1-1 L, 0.1-0.5 L, 1-10 L, 1-9 L, 1-8 L, 1-7 L, 1-6 L, 1-5 L, 1-4 L, 1-3 L, 5-2 L, 5-10 L, 5-9 L, 5- 8 L, 5-7 L or 5-6 L. The fully expanded alveolar airway volume may be about 0.1-7 L. The terminal unit may have a fully compressed airway alveolar volume of about 0.01-5 L, 0.01-4 L, 0.01-3 L, 0.01-2 L, 0.01-1 L, 0.01-0.5 L, 0.01-0.1 L, 0.05-4 L, 0.5-4 L, 0.5-3 L, 0.5-2 L, 0.5-1 L, 1-5 L, 1-4 L, 1-3 L or 1-2 L. The fully compressed alveolar airway volume may be about 0.01-3 L. The terminal unit is in any form, for example, of a sealed hemisphere, bellows, or frustum.

Each lobe compartment may comprise 2-23 (e.g., 2) generation regions. Each generation region may have one or more unique properties, for example, porosity, specific surface area and hydraulic diameter, depending on the lattices in each generation region.

The lattices may have a porosity (E) in the range of about 0.01-1, 0.01-0.5, 0.01-0.1, 0.1-1, 0.1-0.5, 0.1-0.2, 0.5-1, 0.5-0.75, 0.5-0.6, 0.6-0.7, 0.7-0.8, 0.8-0.9 or 0.9-1. The lattices in a generation may have a porosity (E) in the range of about 0.01-1, 0.01-0.5, 0.01-0.1, 0.1-1, 0.1-0.5, 0.1-0.2, 0.5-1, 0.5-0.75, 0.5-0.6, 0.6-0.7, 0.7-0.8, 0.8-0.9 or 0.9-1. The lattices in different generations may have a different porosity (E).

The lattices may have a dimensionless specific surface area (aᵥ·l_{c}) of about 0.01-100, 0.01-50, 0.01-10, 0.01-5, 0.01-1, 0.01-0.5, 0.01-0.1, 0.1-100, 0.1-50, 0.1-10, 0.1-5, 0.1-1, 0.1-0.5, 0.5-100, 0.5-50, 0.5-10, 0.5-5, 0.5-1, 1-100, 1-50, 1-10 or 1-5. The lattices in a generation region may have a dimensionless specific surface area (aᵥ· l_{c}) of about 0.01-100, 0.01-50, 0.01-10, 0.01-5, 0.01-1, 0.01-0.5, 0.01-0.1, 0.1-100, 0.1-50, 0.1-10, 0.1-5, 0.1-1, 0.1-0.5, 0.5-100, 0.5-50, 0.5-10, 0.5-5, 0.5-1, 1-100, 1-50, 1-10 or 1-5. The lattices in different generation regions may have a different dimensionless specific surface area (aᵥ· l_{c}).

The lattices may have a hydraulic diameter (d_{H}) of about 0.01-100, 0.01-50, 0.01-10, 0.01-1, 0.01-0.5, 0.01-0.1, 0.1-100, 0.1-50, 0.1-45, 0.1-40, 0.1-35, 0.1-30, 0.1-25, 0.1-20, 0.1-15, 0.1-10, 0.1-5, 0.1-1, 0.1-0.5, 0.5-100, 0.5-50, 0.5-10, 0.5-1, 1-100, 1-50 or 1-10 mm. The lattices in a generation region may have a hydraulic diameter (d_{H}) of about 0.01-100, 0.01-50, 0.01-10, 0.01-1, 0.01-0.5, 0.01-0.1, 0.1-100, 0.1-50, 0.1-10, 0.1-1, 0.1-0.5, 0.5-100, 0.5-50, 0.5-10, 0.5-1, 1-100, 1-50 or 1-10 mm. The lattices in different generation regions may have a different hydraulic diameter (d_{H}).

The lattices may have a Shore hardness in the range from Shore 00 30 to Shore D 90. The lattices in a generation region may have a Shore hardness in the range from Shore 00 30 to Shore D 90. The lattices in different generation regions may have a different Shore hardness.

The lattices may comprise polymeric periodic porous media compartments. The lattices may comprise unit cells. The unit cells may be symmetric. The unit cells may be asymmetric. The unit cells may be non-uniformed. The unit cells may be patterned. The unit cells maybe non-uniformed and patterned.

Each modular component may be at a distal end of a lobe compartment. The modular component may have a linear rate of compression or expansion at about 0.1-100, 0.1-50, 0.1-10, 0.1-5, 0.1-1, 0.1-0.5, 1-100, 1-50, 1-10, 1-5, 5-100, 5-50, 5-10, 10-100 or 10-50 mm/s.

For each lung replica of the present invention, a method for exchanging airflow in the lung replica is provided. The airflow exchange method comprises compressing each lobe compartment such that an exhaled airflow is generated. The air exchange method further comprises expanding each lobe compartment such that an inhaled airflow is generated. As a result, the lung replica exchanges airflow. The compressing step may be before or after the expanding step. The compressing step and the expanding step may be alternated and repeated.

The airflow exchange method may further comprise holding the inhaled airflow in the lung replica between the expanding step and the compressing step. The inhaled airflow may be held for at least about 0.1, 0.5, 1, 2, 3, 4, 5, 6, 7, 8, 9 or 10 seconds, or about 0.1-10, 0.1-9, 0.1-8, 0.1-7, 0.1-6, 0.1-5, 0.1-4, 0.1-3, 0.1-2, 0.1-1, 0.1-0.5, 0.5-10, 0.5-9, 0.5-8, 0.5-7, 0.5-6, 0.5-5, 0.5-4, 0.5-3, 0.5-2, 0.5-1, 1-10, 1-9, 1-8, 1-7, 1-6, 1-5, 1-4, 1-3, 1-2, 5-10, 5-9, 5-8, 5-7 or 5-6 seconds.

The airflow exchange method may further comprise quantifying the exhaled airflow. The flow rate and/or amount of the exhaled airflow may be measured. The airflow exchange method may further comprise quantifying the inhaled airflow. The flow rate and/or amount of the inhaled airflow may be measured. In the compressing step, each lobe compartment in the lung replica may be fully compressed. In the expanding step, each lobe compartment may be fully expanded.

According to the airflow exchange method, the inhaled airflow may comprise an aerosol, and the aerosol may be deposited in the lung replica. The airflow exchange method may further comprise determining the deposition profile of the aerosol in at least one lobe compartment and/or at least one generation region. The deposit profile of the aerosol may be determined after the exhalation step or the holding step.

The airflow exchange method may further comprise modifying the deposition profile. The deposition profile may be modified by modifying at least one generation region, the lattices, the flow rate and/or amount of the inhaled airflow, the flow rate and/or amount of the exhaled airflow, and/or the duration of the compressing and/or expanding step.

For each lobe compartment according to the present invention, a method for preparation the lobe compartment is provided.

The lobe compartment comprises a lobe airway space and a generation region. The generation region is based on an analogous airway generation in an analogous lobe. The lobe compartment preparation method comprises forming the lobar airway space in the lobe compartment with a series of connected circles having gradually changing diameters along the length of the lobe compartment. The lobe compartment preparation method also comprises adding a lattice into a circle in the connected circles to form a generation region. The generation region has a cross sectional area based on a cumulative cross-sectional area of the analogous airway generation, scaled in inverse proportion to porosity of the lattice. The generation region has a depth based on a length of the analogous airway generation. The lobe compartment preparation method further comprises forming a terminal unit in the lobe compartment. As a result, the lobe compartment is prepared.

The lobe compartment generation method may further comprise repeating lattice addition step for one or more times. The resulting lobe compartment may comprise two or more generations based on two or more analogous airway generations in the analogous lobe. The volume of the lobar airway space may be similar to, for example, about 80-120% of, the airway volume of the analogous lobe.

For each lung replica of the present invention, a method for preparing the lung replica is prepared. The lung replica preparation method comprises providing an upper airway compartment. The upper airway compartment comprises an upper airway space. The lung replica preparation method also comprises providing one or more lobe compartments prepared according the lobe compartment preparation method. The lung replica preparation method further comprises connecting the upper airway space with the lobar airway space in each lobe compartment; and providing one or more modular components. The one or more modular components actuate the one or more lobe compartments to compress or expand, for example, in synchronized movement. An exhaled airflow is generated when the one or more lobe compartments are compressed. An inhaled airflow is generated when the one or more lobe compartments are expanded.

For each lung replica preparation method, a lung replica is prepared. The lung replica comprises an upper airway compartment, one or more lobe compartments, and one or more modular components. The upper airway compartment comprises an upper airway space. Each lobe compartment comprises at least two generation regions, a compressible terminal unit, and lattices in the generation regions. Each lobe compartment comprises a lobar airway space connected with the upper airway space at the proximal end of the lobe compartment. Each modular component actuates one or more lobe compartments to compress or expand such that an airflow is generated. For example, each lobe compartment is actuated by one modular component. An exhaled airflow is generated when the one or more lobe compartments are compressed. An inhaled airflow is generated when the one or more lobe compartments are expanded.

The term “about” as used herein when referring to a measurable value such as an amount, a percentage, and the like, is meant to encompass variations of ±20% or ±10%, more preferably ±5%, even more preferably ±1%, and still more preferably ±0.1% from the specified value, as such variations are appropriate.

### Example 1. Whole Volume Lung Replica

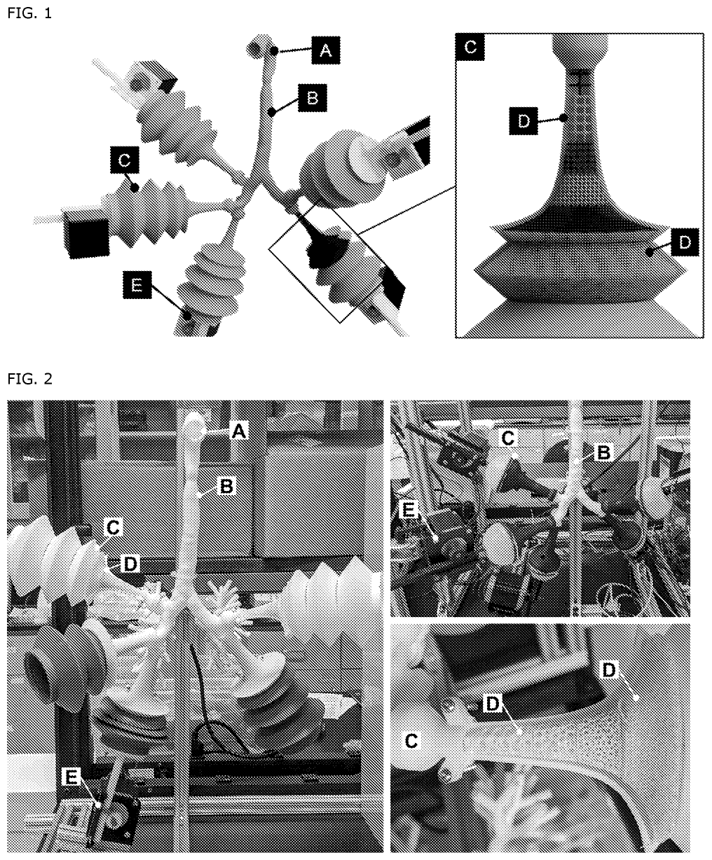

A lung replica as a whole volume lung model has been developed with three key components. The extrathoracic and tracheobronchial airways (FIGS. 1A and B) may be patient-specific or idealized. Each of the detachable and sealed lobes (FIG. 1C) includes a compressible terminal unit. The lobes include removable lattice inserts. The breath function may be accomplished by actuators for the lobe ends. The spatial deposition within the lobe regions may be quantified.

FIG. 2 shows realization of the whole volume model. A cross section of a full volume adult lobe compartment (C) is shown to the right (bottom), where internal lattice elements (D) are indicated. D indicates removable lattice inserts within the lobe compartment. The two D regions selected have differing levels of porosity. E indicates a component which mechanically actuates the deformable unit, which is present for all 5 lobe units in the full invention.

The inventors have successfully realized a 5-lobe prototype scaled for an idealized adult male (FIG. 2, left) and a 5-lobe prototype scaled for an idealized” small-volume” prototype (FIG. 2, top right) that is fully sealed and functional.

Components A and B represent the extrathoracic and tracheobronchial airway compartments and were derived from a validated upper airway from a CT scan of a healthy volunteer (male, age 47). They were segmented in Materialise Mimics and manipulated in Solidworks to have a 2 mm wall thickness for a representative idealized upper airway per standard techniques. (See Feng et al., An In Silico Subject-Variability Study of Upper Airway Morphological Influence on the Airflow Regime in a Tracheobronchial Tree. Bioengineering. 2017; 4 (4): 90. Doi: 0.3390/bioengineering4040090. PubMed PMID: 29144436; Kolewe et al., Realizing Lobe-Specific Aerosol Targeting in a 3D-Printed In Vitro Lung Model. J Aerosol Med Pulm Deliv. 2020. Doi: 10.1089/jamp.2019.1564). The lobar bronchi were truncated and modified at the outlet to interface with the lobe unit in CAD. Components A and B were printed as a continuous part using the M1 printer in the non-deformable PR 25 resin. The part is the same in both the adult and small-volume prototype.

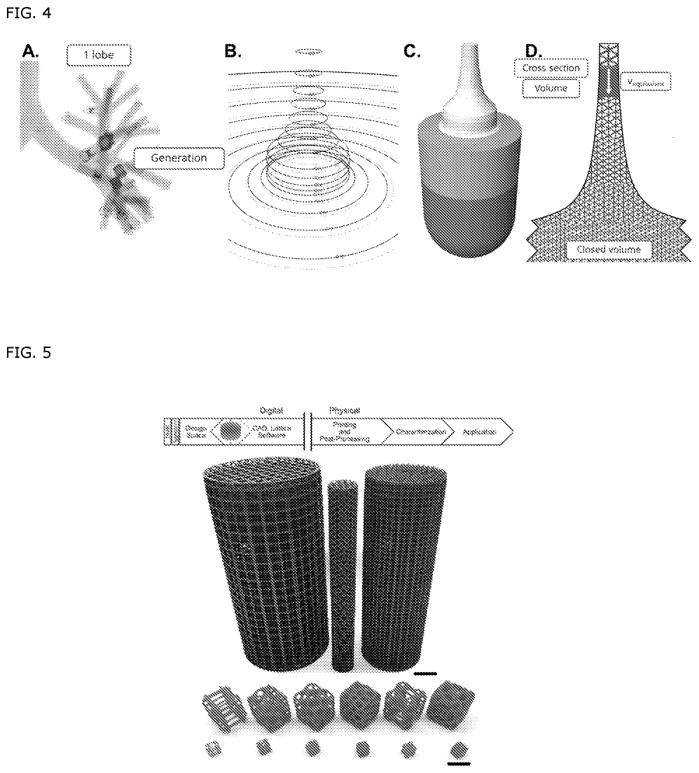

The lobe unit or component C (FIG. 1) for the adult prototype replica model has been designed (FIG. 4C). In this idealized adult male case, the FRC and lobar volume distribution are taken from published values of idealized adult male, including the Weibel morphometric model. (Weibel, E. R. Morphometry of the Human Lung. (Springer Berlin Heidelberg, 1963). doi: 10.1007/978-3-642-87553-3; Weibel and Gomez, Architecture of the human lung. Use of quantitative methods establishes fundamental relations between size and number of lung structures. Science. 1962; 137 (3530): 577-85. doi: 10.1126/science.137.3530.577. PubMed PMID: 14005590); “Weibel and Gomez (1962)”). For each generation, the cumulative cross-sectional area was calculated by summing the cross sectional area of all airways in the given generation. The generation of terminal bronchioles was taken from the applied morphometric model for each lobe, and a trumpet/horn combined airway replica was generated for the generations starting from the lobe entrance (G2/3) up to the generation of the terminal bronchioles and up to three generations past, where the respiratory bronchioles become fully alveolated. The general structure of the horn replica is such that each generation was represented by a circle of equivalent area as the cumulative cross-sectional area for the generation, and the spacing between one generation and the next was equal to the length of the parent generation. After the final combined generation, all remaining airways before the alveoli are represented by extruding the circle of the final generation out to an equivalent volume (FIG. 4C).

To create the manufacturable shell of a horn replica, a 0.5-5 mm wall thickness was added to a surface created by smooth transition (loft) between the diameters of the generation equivalents and the extrusion of the final generation. The diameter of the first approximated generation was adjusted to match or exceed the circumscribed outlet airway diameter. A mate fitting and corresponding mounting posts were added to fit the lobe shell to the airway outlet at the tracheobronchial tree. At the base of the shell, a groove for an o-ring was cut into the shell. The lobe approximation shells was produced by additive manufacturing on the Carbon M1 printer.

On the small volume prototype (FIG. 2 top right), we demonstrate an enclosed form of equal volume of terminal alveoli, which takes the form of a hemisphere, bellows, or frustum (referred to as the alveolar volume). The shape negative was designed in a multi-part assembly to create a mold of the enclosed form, from which the volume was cast from a silicone, with Shore hardness in the range of Shore 00 30 to Shore A 30. The elastic properties here are essential to result in both compression and tension of the part. After casting, the base of the alveolar volume was fitted with an attachment point for actuation by the motion control system.

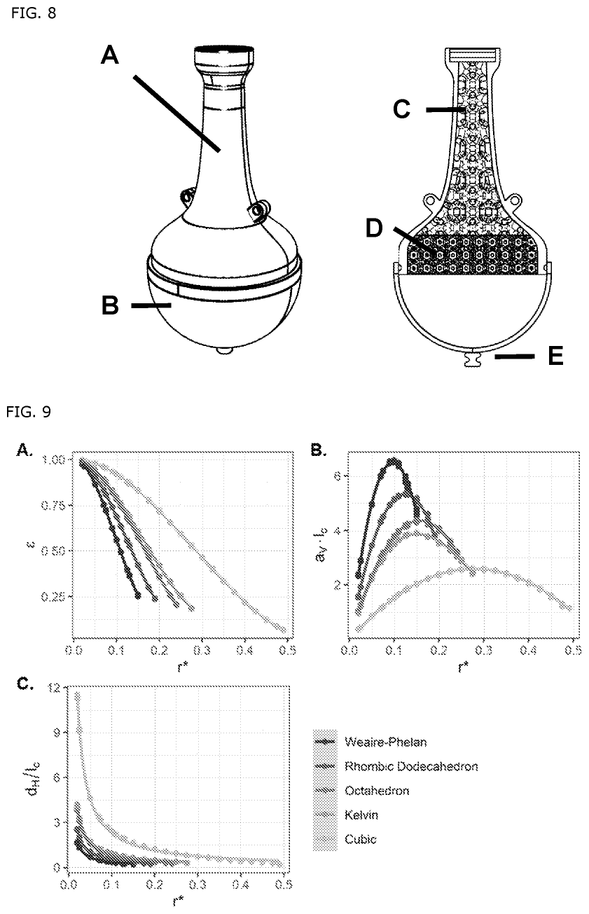

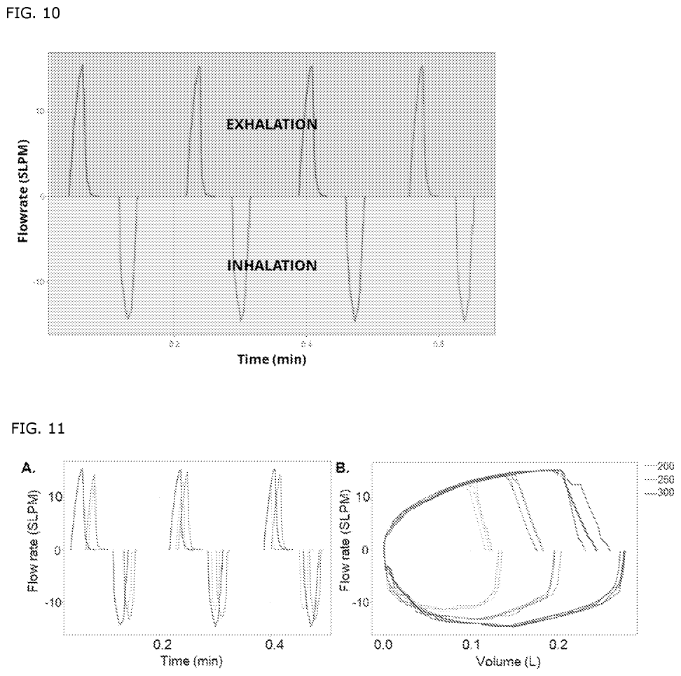

Lattice components D (FIG. 1, FIG. 2, FIG. 8) were generated using the software package Rhino 6 (Robert McNeel & Associates) and Grasshopper plugin, per standard protocols and recently published works. (See Woodward and Fromen, Scalable, process-oriented beam lattices: Generation, characterization, and compensation for open cellular structures. Additive Manufacturing. 2021; 48:102386). Lattice design regions within a lobe unit were grouped together based on an arbitrary 2 generations for each removable lattice part. Each grouping of lattices was printed piecewise, using commercially available resins (PR25, UMA90), cleaned per manufacturer's instructions. Lattice designs in the prototype (FIG. 8) are demonstrated with decreasing hydraulic diameter, corresponding to increasing deposition efficiency expected in the lung with increased generation depth.

The lung replica may be adjusted to achieve desirable appropriate resolution of its components. The internal surfaces of the lattices are important to ensuring deposition and do not promote aerosol bounce; as-printed parts made of all 3D printed resins have been acceptable. Coating of lattices through thin films to mimic the mucous interface of the lung and/or oxygen plasma treatment can be pursued; this can help remove deposited aerosols during the washing step. The materials are compatible with a washing step and downstream analytical approach. The deformable sealed components of the lung must result in both compression and expansion; we have used a silicone, with Shore hardness in the range of Shore 00 30 to Shore A 30.

### Example 2. Truncation of an Airway Tree Based on the Centerline of Each Airway

Available airways are isolated from an X-ray computed tomography (CT) scan, magnetic resonance image (MRI), or other medical imaging output, when available for an individual patient. The airways are converted to a surface representation, such as a stereolithography (STL) file. In an alternative case, the extrathoracic airways (mouth, nose, throat) are replaced with an idealized representation. In an alternative case, an idealized tracheobronchial tree is generated from a scaled morphometric model (for example, ICRP, 1994. Human Respiratory Tract Model for Radiological Protection. ICRP Publication 66. Ann. ICRP 24 (1-3); Weibel, E. R. Morphometry of the Human Lung. (Springer Berlin Heidelberg, 1963). doi: 10.1007/978-3-642-87553-3; Models of human lung airways and their application to inhaled particle deposition. Bull Math Biol. 1980; 42 (3): 461-80 doi: 10.1007/bf02460796. PubMed PMID: 7378614; Soong TT, Nicolaides P, Yu CP, Soong SC. A statistical description of the human tracheobronchial tree geometry. Respir Physiol 1979; 37:161-72. DOI: 10.1016/0034-5687 (79) 90068-9. PMID: 472520; Phalen RF, Oldham MJ, Beaucage CB, Crocker TT, Mortensen J. Postnatal enlargement of human tracheobronchial airways and implications for particle deposition. Anat Rec 1985; 212:368-80. DOI: 10.1002/ar.1092120408. PMID: 4073554.). The functional residual capacity is assumed and used to scale morphometric dimensions by the cube root of the ratio of the assumed FRC to the model FRC. From the resulting full lung description, a skeleton of the upper airways is generated and filled to the respective volume of the scaled morphometric volume for each generation. The resulting surface may be used in an analogous process to the patient-derived replica.

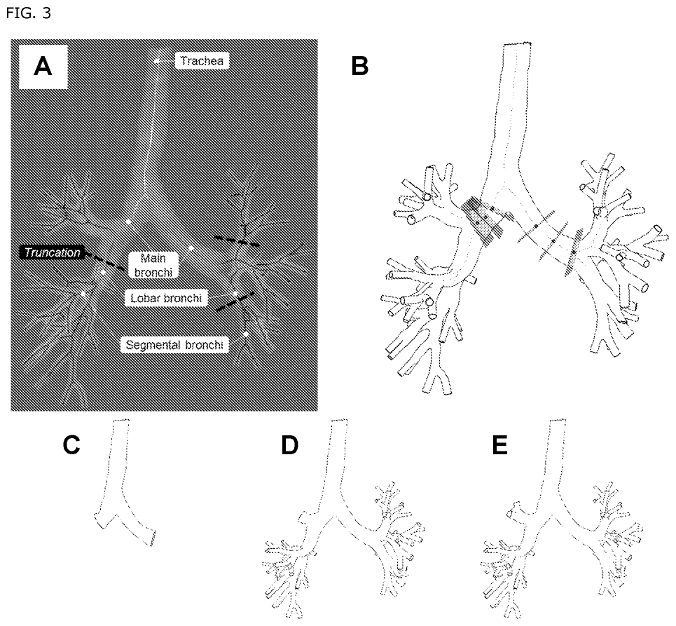

In all cases, once converted to a surface model, the airways were skeletonized to identify the medial axis or centerline of each airway (FIG. 3), as well as the distance transform or maximum inscribed sphere radius. These outputs effectively describe the length, diameter, and connectivity of each airway. With this information, the lobar bronchi were isolated and truncated distally at a point 50-100% along the length of the respective centerline. Representative truncation sites of the main bronchi (FIG. 3C), right upper lobar bronchi (FIG. 3D), and right upper segmental bronchi (FIG. 3E) are indicated. If segmented at the main bronchi, a full replica would result in 2 divisions and require 2 lobe units. If segmented at the lobar bronchi, a full replica would result in 5 divisions and require 5 lobe units. If segmented at the segmental bronchi, a full replica would result in 18 divisions and require 18 lobe units. The cutting plane is oriented with a normal vector aligned with the centerline of the truncated airway, and the proximal section of the surface model is retained from each cut. This corresponds to the parts shown in FIG. 2.

### Example 3. Generation of Lobe Units from Airway Data

A lobe unit is based on a horn structure and is generated for the airway generations starting from the lobe entrance (G2/3) up to the generation of the terminal bronchioles and up to three generations past, where the respiratory bronchioles become fully alveolated.

For each airway generation (FIG. 4A), the cumulative cross-sectional area is calculated by summing the cross sectional area of all airways in the given generation. This cross-sectional area can come from morphological models or from individual patient data. The average length of each generation is then measured. This can similarly be obtained from morphological models or from individual patient data. These two values, obtained for each generation within a given lobe (or averaged across the entire lung depending on the information source) are used to generate the lobe horn structure.

The general structure of the horn replica is such that each generation is represented by a circle of equivalent area as the cumulative cross-sectional area for the generation, and the spacing between one generation and the next is equal to the length of the parent generation. These are visualized in FIG. 4B. These are then combined sequentially, resulting in a gradually increasing diameter along the length of the lobe unit, as shown in FIG. 4C.

After the final combined generation, all remaining airways before the alveoli are represented by extruding the circle of the final generation out to an equivalent volume that is sealed. FIG. 4C shows a representative the full lobe design generated by this method.

The total volume, once populated with lattices, should match the target total lung capacity (either average or individual patient value). The cross-sectional diameter can be scaled to accommodate the additional space taken by the lattice structures.

Tables 1-4 detail the relevant quantities used in defining the approximation geometry shown in our preliminary data. These are taken from Yeh & Schum (1980) (Models of human lung airways and their application to inhaled particle deposition. Bull Math Biol. 1980; 42(3):461-80 doi: 10.1007/bf02460796. PubMed PMID: 7378614) as an average morphometric data set to demonstrate the overall method. In the following tables, entries that are initialized entries indicate source lung data, while non-italicized represent obtained model replica features (either calculated or design choice).

TABLE 1
Morphometric lung measurements from Yeh & Schum (1980)

| | | | | | | Cross | | Cumulative |
|---|---|---|---|---|---|---|---|---|
| | | Length | Diameter | Branching | Gravity | Section | Volume | Volume |
| Generation | Tubes | (cm) | (cm) | Angle (°) | Angle(°) | (cm²) | (cm³) | (cm³) |
| 1 | 1 | 10 | 2.01 | 0 | 0 | 3.17 | 31.73 | 31.73 |
| 2 | 1 | 5.63 | 1.38 | 40 | 15 | 1.5 | 8.42 | 40.15 |
| 3 | 1 | 1.42 | 1.15 | 25 | 10 | 1.04 | 1.47 | 41.62 |
| 4 | 2 | 1.33 | 0.905 | 48 | 55 | 1.29 | 1.71 | 43.33 |
| 5 | 4 | 1.13 | 0.68 | 25 | 48 | 1.45 | 1.64 | 44.97 |
| 6 | 8 | 0.891 | 0.559 | 31 | 41 | 1.96 | 1.75 | 46.72 |
| 7 | 16 | 1.02 | 0.454 | 22 | 39 | 2.59 | 2.64 | 49.36 |
| 8 | 32 | 0.836 | 0.365 | 27 | 37 | 3.35 | 2.8 | 52.16 |
| 9 | 64 | 0.778 | 0.316 | 27 | 36 | 5.02 | 3.91 | 56.07 |
| 10 | 128 | 0.771 | 0.298 | 28 | 36 | 8.93 | 6.88 | 62.95 |
| 11 | 256 | 0.611 | 0.286 | 31 | 46 | 16.45 | 10.05 | 73 |
| 12 | 512 | 0.544 | 0.211 | 40 | 45 | 17.9 | 9.74 | 82.74 |
| 13 | 1024 | 0.431 | 0.146 | 46 | 48 | 17.14 | 7.39 | 90.13 |
| 14 | 2048 | 0.302 | 0.102 | 50 | 60 | 16.73 | 5.05 | 95.18 |
| 15 | 4096 | 0.224 | 0.076 | 52 | 60 | 18.58 | 4.16 | 99.34 |
| 16 | 8192 | 0.188 | 0.061 | 54 | 60 | 23.94 | 4.5 | 103.84 |
| 17 | 16384 | 0.152 | 0.055 | 45 | 60 | 38.93 | 5.92 | 109.76 |
| 18 | 32768 | 0.124 | 0.051 | 45 | 60 | 66.94 | 8.3 | 118.06 |
| 19 | 65536 | 0.103 | 0.049 | 45 | 60 | 123.58 | 12.73 | 130.79 |
| 20 | 131072 | 0.087 | 0.047 | 45 | 60 | 227.4 | 19.78 | 150.57 |
| 21 | 262144 | 0.076 | 0.046 | 45 | 60 | 435.66 | 33.11 | 183.68 |
| 22 | 524288 | 0.066 | 0.045 | 45 | 60 | 833.84 | 55.03 | 238.71 |
| 23 | 1048576 | 0.059 | 0.044 | 45 | 60 | 1594.39 | 94.07 | 332.78 |
| 24 | 2097152 | 0.053 | 0.043 | 45 | 60 | 3045.49 | 161.41 | 494.19 |
| 25 | 92300000 | 0.025 | 0.03 | 45 | 60 | NA | 1191.22 | 1685.41 |

TABLE 2
Overall lobe properties for approximation
strategy, based on Yeh & Schum (1980)

| Lobe Position | Left Lower |
|---|---|
| Lobe Volume (mm³) | 1685410 |
| Alveolar Volume (mm³) | 1191220 |
| Generation Numbering | Weibel (1962) |
| Source | |
| Approximation Start | 3 |
| Generation | |
| Terminal Bronchiole | 15 |
| Generation | |
| Terminal Generation Offset | 2 |

TABLE 3
Airway properties for approximation strategy, based on derived
quantities from morphometric data for the left lower lobe (Yeh & Schum,
1980).⁸ Generation numbering scheme is taken from Weibel (Weibel,
E. R. Morphometry of the Human Lung. (Springer Berlin Heidelberg,
1963). doi: 10.1007/978-3-642-87553-3.)

| | | Generation | Equivalent | Pre-scaled |
|---|---|---|---|---|
| | Length | Volume | Cross-sectional | Approximation |
| Generation | (mm) | (mm³) | Diameter (mm) | Diameter (mm) |
| 0 | 100 | 31730 | 20.09 | — |
| 1 | 56.3 | 8420 | 13.82 | — |
| 2 | 14.2 | 1470 | 11.51 | — |
| 3 | 13.3 | 1710 | 12.82 | 12.82 |
| 4 | 11.3 | 1640 | 13.59 | 13.59 |
| 5 | 8.91 | 1750 | 15.8 | 15.8 |
| 6 | 10.2 | 2640 | 18.16 | 18.16 |
| 7 | 8.36 | 2800 | 20.65 | 20.65 |
| 8 | 7.78 | 3910 | 25.28 | 25.28 |
| 9 | 7.71 | 6880 | 33.72 | 33.72 |
| 10 | 6.11 | 10050 | 45.77 | 45.77 |
| 11 | 5.44 | 9740 | 47.74 | 47.74 |
| 12 | 4.31 | 7390 | 47.74 | 47.74 |
| 13 | 3.02 | 5050 | 47.74 | 47.74 |
| 14 | 2.24 | 4160 | 48.64 | 48.64 |
| 15 | 1.88 | 4500 | 55.21 | 55.21 |
| 16 | 1.52 | 5920 | 70.4 | 70.4 |
| 17 | 1.24 | 8300 | 92.32 | 92.32 |
| 18 | 1.03 | 12730 | 125.44 | 92.32 |
| 19 | 0.87 | 19780 | 170.16 | 92.32 |
| 20 | 0.76 | 33110 | 235.52 | 92.32 |
| 21 | 0.66 | 55030 | 325.83 | 92.32 |
| 22 | 0.59 | 94070 | 450.56 | 92.32 |
| 23 | 0.53 | 161410 | 622.71 | 92.32 |

TABLE 4
Lattice properties for approximation strategy, based on a two-
lattice-region lobe design with generation-weighted hydraulic
diameter taken as criterion for lattice parameter selection.

| | | Hydraulic | | Cell | | |
|---|---|---|---|---|---|---|
| Start | End | diameter | Unit | Dimension | Dimensionless | Ideal |
| Generation | Generation | (mm) | Cell | (mm) | radius | Porosity |
| 3 | 15 | 3.665 | Weaire- | 9.578 | 0.09 | 0.6246 |
| | | | Phelan | | | |
| 15 | 23 | 0.4975 | Weaire- | 1.3 | 0.09 | 0.6246 |
| | | | Phelan | | | |

### Example 4. Lattices

We have established an in house framework for generating uniform lattices and conformal lattice skins without need of advanced meshing techniques (FIG. 5). We have printed these structures using the digital light processing (DLP) variant offered by the Carbon M1, continuous liquid interface production (CLIP™) (see Tumbleston et al., Additive manufacturing. Continuous liquid interface production of 3D objects. Science. 2015; 347 (6228): 1349-52. Epub 2015-3-18. doi: 10.1126/science.aaa2397. PubMed PMID: 25780246) and have developed design-stage functional grading approaches to address curing artifacts with a generalizable procedure that is broadly applicable for a range of parts and systems. Enabled by this capability, we have generated complex lattice designs of a wide range of unit cell geometries and total length scales. Physical parts shown in FIG. 5 were created from this method and printed in UMA90 resin and washed per manufacture's recommendations. FIG. 5 demonstrates lattices of a large range of part dimensions and lattice geometries.

### Example 5. Directional Lattices

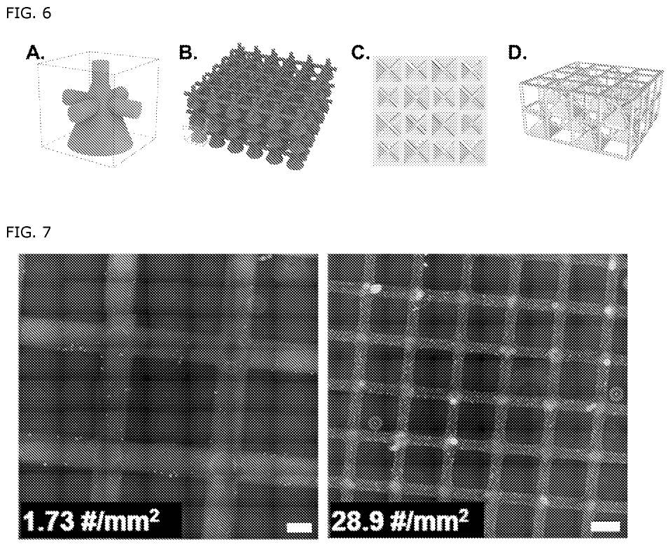

Building on our in house lattice framework, we have also demonstrated creation of directional lattices that will inspire different flow properties depending on the flow conditions. FIGS. 6A and B demonstrate periodic uniform lattices (all unit cells are the same), while FIGS. 6C and D demonstrate patterning of some directional and some open lattices. The overall patterning can be well controlled throughout the part to further impose flow directionality within a given lattice.

This design is important as it can provide opportunities to regionally change aerosol deposition efficiency depending on direction of the flow. For example, these lattices can have increased deposition efficiency in the lung replica under inhalation, but lower efficiency when the flow is reversed during exhalation.

### Example 6. Aerosol Deposition

An in-house flow apparatus has been established to evaluate air and aerosol transport through lattice materials of different sizes (FIG. 7). Clear pipes approximately 0.5 m in length, 34.1 mm in diameter, were mounted vertically with a removable fitting at the bottom for inserting lattice samples. Once lattices were placed inside the sample region, the pipe was connected in line with a mass flow meter (FT, TSI Model 4043), flow controller (FC, Copley TPK 2000), and vacuum pump (Copley HCP5). An optical particle sizer (OPS 3330 TSI), a portable spectrometer, was attached to the pipe for upstream and downstream aerosol concentration measurements obtained in real time.

Once flow conditions were calibrated, aerosols were introduced at the pipe inlet and allowed to deposit over the experimental cycle. Monodisperse fluorescent polystyrene particles 1 μm in diameter were dispersed from a collision jet nebulizer (CJN) from methanol to ensure uniform aerosol generation. Aerosols were deposited independently on two uniform stack of cubic lattices, with unit cell opening sizes of 3.5 mm (left) and 1.3 mm (right), under a 15 LPM flow rate and visualized via fluorescence microscopy. Aerosol deposition was quantified through fluorescence imaging on the accessible part surfaces. From this experiment, we observed uniform spatial deposition across both cubic unit cells and demonstrate that the larger 3.5 mm unit cell averages˜1.73 particles/mm² while the smaller 1.3 mm unit cell averages 28.9 particles/mm².

These results are important in establishing that varied geometries of lattices will generate varied aerosol deposition patterns. Selection of the appropriate lattice geometry is thus critical in recreating the local deposition patterns that occur in the lung.

### Example 7. Test Lobe Assembly

A test lobe assembly was designed for a small-scale lung volume and populated with two lattice regions. The upper (proximal) lattice region was designed to approximate the average hydraulic diameter of generations 4-10 (Table 1) with a Weaire-Phelan unit cell; cell length, I_{c}=13.93 mm; and dimensionless radius, r*=0.09. The lower (distal) lattice region was designed to approximate the average hydraulic diameter of generations 11-13 (Table 1) with a Weaire-Phelan unit cell; cell length, I_{c}=5.73 mm; and dimensionless radius, r*=0.09. These configurations yield respective hydraulic diameters within an ideal single unit cell of approximately 5.3 mm and 2.1 mm. The alveolar volume of the test lobe assembly was approximately 70 mL.

The parts for the test lobe assembly were assembled and mounted vertically (FIG. 8, right) on a ring stand clamp in a fume hood. An AERONEB® lab nebulizer with flow rate>0.1 mL/min and aerosol volumetric median diameter (VMD) of 2.5-4.0 μm was loaded with 100 μL of a solution containing 100 mg/ml of fluorescent dye Rhodamine B. The nebulizer was connected to the lobe in series using a Y-shaped adapter, with the other side of the adapter partially closed with an exhaust collection chamber to capture any particles “exhaled” by the lobe motion. The nebulizer was powered on, and over a period of 40 seconds, the test lobe assembly was manually actuated (fully compressed and released) approximately every 4 seconds. After conducting the trial, the test assembly and delivery components (nebulizer, adapter, and exhaust chamber) were washed with water, the solvent was analyzed for fluorescent signal, and the relative deposition amounts were quantified based on a standard curve of Rhodamine B in water. Approximately 81% of the originally loaded Rhodamine B was accounted for, and the ratio of deposited aerosol in the proximal vs distal lattice was approximately 3:1, reported in Table 5.

TABLE 5
Results of test lobe deposition trial. A quantification
of the total percentage of Rhodamine B collected from
the initial amount loaded into the nebulizer (Total
Collected) and the distribution of the Deposited Dose
In each component is shown. Deposited Dose was calculated
by adding the mass collected on components A-D.

| | | Total | Deposited |
|---|---|---|---|
| | Components | Collected (%) | Dose (%) |
| A, B | Lobe shell and alveolar shell | 6 | 23 |
| C | Proximal lattice | 15 | 58 |
| D | Distal lattice | 5 | 19 |
| | Nebulizer | 27 | — |
| | Adapter | 22 | — |
| | Exhaust collection chamber | 6 | — |

### Example 8. Geometric Properties of Lattices

Lattices of different unit cell geometry can yield a range of different relevant properties needed in the selection of the geometry for the lung replica. FIG. 9 demonstrates how important selection criteria vary with the dimensionless strut radius (r*) and a given unit cell geometry. Five representative unit cell geometries were chosen (Weaire-Phalen, Cubic, Kelvin etc.) to demonstrate the overall design space available. These unit cell geometries are well characterized and afford a range of achievable features. While selections of hydraulic diameter, porosity, and surface area may all be pursued to select these features of the given airway generation groups, hydraulic diameter (d_{H}) was used in our preliminary data. Once a d_{H} and r* are prescribed for a given unit cell geometry, the other relevant properties will be defined as shown in this figure.

### Example 9. Repeatable Breathing Profiles of Lung Replica

Using the 5-lobe prototype scaled for an idealized small volume (FIG. 2 top right) that was fully sealed and functional, we have demonstrated capacity for measuring breathing profiles for the lung replica. The 5-lobe prototype was made as per our prior description in FIG. 2 and attached to 5 stepper motors controlled via an in-house Arduino program, which operated the speed and distance of the stepper. The replica prototype was maintained in a fixed position attached to a frame, with each lobe aligned with its designated motor, beginning from an open, uncompressed position. Motor actuation compressed the deformable lobe unit to generate an exhalation maneuver; reversing the process generated an inhalation maneuver as the lobe units re-inflate. The overall flow rate was measured at the mouth inlet via flow meter (TSI 4043). The plot shows the flow rate (SLPM) vs time (min) as measured at the mouth of the sealed 5-lobe prototype. The process was repeatable, as shown by the 4 representative profiles, with all 5-lobes deformed under synchronized movement. This data is important in demonstrating the repeatability and overall breathing capacity of the fully sealed 5-lobe replica.

### Example 10. Flow Control in Lung Replica

Using the 5-lobe prototype scaled for an idealized small volume (FIG. 2 top right) that was fully sealed and functional, we have demonstrated capacity for maintaining a peak inlet flow rate while exchanging varied volumes of lobe fraction (FIG. 11). The flow rate was measured at the mouth inlet via flow meter (TSI 4043). The series shown (200, 250, 300) indicate the number of steps taken by the motor, corresponding to increasing volumes of air exchanged. By tuning the rate of the steps, the total flow rate was controlled to match the same peak volume. The process was repeatable, as shown by the 3 representative profiles, taken with all 5-lobes deformed under synchronized movement. The slight off-set shown in B between profiles was due to an imposed directional sampling error of the TSI 4043 used to measure the flow rate at the mouth.

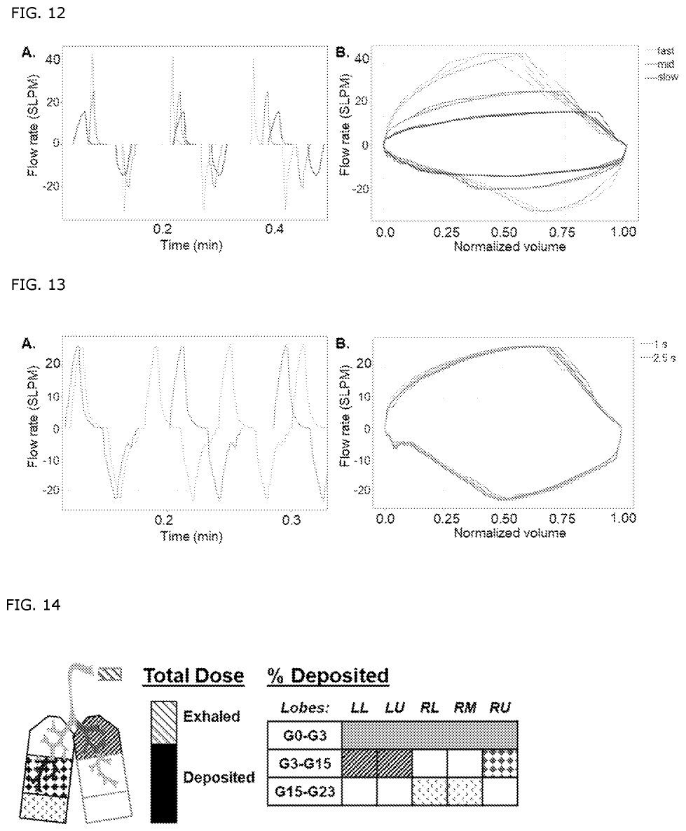

Using the 5-lobe prototype scaled for an idealized small volume (FIG. 2 top right) that was fully sealed and functional, we have demonstrated capacity for maintaining a fixed volume exchange while generating varied peak flow rates (both inhalation and exhalation) (FIG. 12). The series shown (fast, mid, slow) indicates the rate of steps taken by the motor, corresponding to increasing speeds to generate varied peak flow rates. By compressing and expanding each lobe unit to the same fraction in each maneuver, the total volume was fixed. The process was repeatable, as shown by the 3 representative profiles, taken with all 5-lobes deformed under synchronized movement.

Results from FIGS. 11 and 12 demonstrate key criteria in re-creating breathing maneuvers of the human lung.

### Example 11. Breath Holds in Lung Replica

Using the 5-lobe prototype scaled for an idealized small volume (FIG. 2 top right) that was fully sealed and functional, we have demonstrated capacity for maintaining a fixed volume exchange while generating varied peak flow rates (both inhalation and exhalation) (FIG. 13). The series shown (1 s, 2.5 s) indicates the time between breathing maneuvers, corresponding to the duration of breath hold generated by the replica. The process was repeatable, as shown by the 3 representative profiles, taken with all 5-lobes deformed under synchronized movement. These results highlight a critical innovation in re-creating breathing maneuvers of the human lung. Breath holds are known to be critical in increasing airway deposition; other experimental models are unable to quantitatively evaluate the role of breath hold duration in airway deposition efficiency, which will be enabled by these results.

### Example 12. Quantification of Deposited Aerosol in Lung Replica

FIG. 14 qualitatively describes the outputs that will be generated from the proposed replica model. Under an imposed set of conditions (model geometry, breathing maneuver), regional deposition will be quantified within each of the 5 lobes at every lattice generational position, as well as the amount exhaled. Quantification depends on the aerosol, but uses standard chemical and imaging-based techniques available. From this regional quantification at each position, the deposited and exhaled fraction can be determined from the total aerosol dosed, as well as the percent deposited within each local generational region. Experimental replicates will be performed to determine the degree of variability under the imposed conditions. Breathing conditions can be iterated over the same geometric model to further identify how deposition changes with the varied breathing conditions.

Using this spatially resolved information described in FIG. 14, inhaler devices/spacers/formulations can be customized and tested to improve deposition to target regions of the lung. The identified target conditions will be determined based on clinical needs to increase local action at the needed site (and may include a set of generations or a particular coordinate within the lung, i.e. left lower, generation 8). The new aerosol input is tested on the replica under a range of breathing conditions and spatial profile measured for each condition. The user can then identify the optimal breathing profile needed to achieve target deposition with the new device, and iterate on the aerosol input features to improve deposition to the target site.

This spatial resolution can take into account patient lobe-level impairments to determine if changes to dosing (via new inhaler, breathing pattern etc.) can improve local accumulation. For example, if one lobe has abnormally low airway volumes; that information can be included in the replica model design prior to inhaler testing. The process is repeated above, where changes to the aerosol input (device/formulation/spacer etc.) are optimized to increase target deposition based on the outputs of this model. The invention is then an important step in the iterative design process, taking into account patient-specific features.

Spatially resolved information can also be used to determine the source and extent of exposure hazards; certain regions of the lung will have higher biological implications. Here, identifying regional deposition under imposed representative breathing conditions (i.e., heavy exercise or sleep) can be used to quantify the total amount deposited and correlate to clinical outcomes.

### Example 13. Lung Replica

### A. Method to Create a Lung Replica

### Lung Replica

A full lung description includes dimensions (length, diameter, branching and gravity angles, number, and volume) of the tracheobronchial, intermediate, and pulmonary airways in a lung. Furthermore, it includes relevant quantities such as total lung capacity (TLC), functional residual capacity (FRC), vital capacity (VC), and possibly residual volume and inspiratory reserve volume. These metrics should be included at minimum for upright position and other positions and environmental conditions as relevant for aerosol deposition studies. A full lung description further includes quantification of volume distributed to each of the five main lobes or each of the 18 lobar segments across the left and right lungs.

Given a full lung description, which is obtainable at an individual or population-level via other methods, the inventors have generated a lung replica using periodic porous media to approximate distal airways beyond the lobar or segmental bronchi. The lung replica may consist of fully-replicated airways up the truncated bronchi (i.e., extrathoracic and tracheobronchial airways). Beyond the fully-replicated airways, a lobe region of equivalent volume as the lung description may encapsulate a porous substrate of two or more forms. The lobe region may be closed, yielding a common inlet and outlet connected to the lung replica at its respective lobar or segmental bronchus. In the case of two forms of porous media, the regions may correspond to central and peripheral regions of the lung, while the cases of two or more regions correspond to groupings of airway generations. Each generational grouping may include as few as one or as many as 23 generations from the complete lung description. Groups within each lobe unit may correspond to biological regions (e.g., bronchi, bronchioles, terminal bronchioles, respiratory bronchioles, alveolar ducts and alveoli), historically used regions in deposition modeling (e.g., bronchial, bronchiolar and alveolar), or algorithmically determined groups determined by artificial intelligence/machine learning clustering of airways by length and diameter via an appropriate algorithm (e.g., K-means, Gaussian Mixture). Between-group divisions occur at points along the main axis of a lobe approximation corresponding to a summed length of the contained airway generations. Further division may take place within groups at regular intervals in the radial or circumferential directions, such that when assembled, all pieces conform to the lobe shell form.

Due to the challenges in obtaining a full lung description for a single patient or a population, patient data, correlations, statistical data, and morphometric lung models may be combined or augmented reciprocally to obtain a full lung description. In this case, quantities of interest may be scaled using methods reported previously to obtain a suitable augmented dataset inclusive of all quantities necessary for a full lung description.

The process for generating the airway tree is as follows:

Available airways may be isolated from an X-ray computed tomography (CT) scan, magnetic resonance image (MRI), or other medical imaging output. The airways may be converted to a surface representation, such as a stereolithography (STL) file. Once converted to a surface model, the airways may be skeletonized to identify the medial axis or centerline of each airway (FIG. 3), as well as the distance transform or maximum inscribed sphere radius. These outputs may effectively describe the length, diameter, and connectivity of each airway. With this information, the lobar bronchi may be isolated and truncated distally at a point 50-100% along the length of the respective centerline. The cutting plane may be oriented with a normal vector aligned with the centerline of the truncated airway, and the proximal section of the surface model may be retained from each cut. If the truncation operation would eliminate critical airway features, the cutting plane may be reoriented appropriately. The surface of the truncated model may be then thickened by 0.5-3 mm to create a solid form. At each bronchial airway outlet, a circular fitting of nominal diameter up to 30 mm may be oriented in alignment with the airway outlet and joined to the replica model. The fitting may be of any type effective for the medium-screw, press-fit, face-seal, or clamp. Proximal to the fitting, mounting posts are affixed to the solid replica model. The upper airway model may be then made by additive manufacturing methods.

In an alternative case, the extrathoracic airways (mouth, nose, throat) may be replaced with an idealized representation.

In an alternative case, an idealized tracheobronchial tree may be generated from a scaled morphometric model (for example, ICRP, 1994. Human Respiratory Tract Model for Radiological Protection. ICRP Publication 66. Ann. ICRP 24 (1-3); Weibel, E. R. Morphometry of the Human Lung. (Springer Berlin Heidelberg, 1963). doi: 10.1007/978-3-642-87553-3; Models of human lung airways and their application to inhaled particle deposition. Bull Math Biol. 1980; 42 (3): 461-80 doi: 10.1007/bf02460796. PubMed PMID: 7378614; Soong TT, Nicolaides P, Yu CP, Soong SC. A statistical description of the human tracheobronchial tree geometry. Respir Physiol 1979; 37:161-72. DOI: 10.1016/0034-5687 (79) 90068-9. PMID: 472520; Phalen RF, Oldham MJ, Beaucage CB, Crocker TT, Mortensen J. Postnatal enlargement of human tracheobronchial airways and implications for particle deposition. Anat Rec 1985; 212:368-80. DOI: 10.1002/ar.1092120408. PMID: 4073554.). The functional residual capacity may be assumed and used to scale morphometric dimensions by the cube root of the ratio of the assumed FRC to the model FRC. From the resulting full lung description, a skeleton of the upper airways may be generated and filled to the respective volume of the scaled morphometric volume for each generation. The resulting surface may be used in an analogous process to the patient-derived model.

In an alternative case, a procedural texture may be applied to the idealized tracheobronchial tree to mimic biological surface features.

In an alternative case, the tracheobronchial airways may be truncated at the segmental bronchi, and lobe approximations are generated for each lobe segment.

The process for generating the lobe approximation is as follows:

If a full lung description is not available, scaling may be applied to the available airway dimensions from patient data or morphometric models by applying a scale factor of the cube root of the ratio of target FRC to morphometric model FRC. Furthermore, the lobar volume distribution may be taken from medical image data or applied from published values. For each generation, the cumulative cross sectional area may be calculated by summing the cross sectional area of all airways in the given generation. The generation of terminal bronchioles may be taken from the applied morphometric model for each lobe, and a trumpet/horn combined airway model may be generated for the generations starting from the lobe entrance (G2/3) up to the generation of the terminal bronchioles and up to three generations past, where the respiratory bronchioles may become fully alveolated. The general structure of the horn replica may be such that each generation may be represented by a circle of equivalent area as the cumulative cross sectional area for the generation, and the spacing between one generation and the next may be equal to the length of the parent generation. After the final combined generation, all remaining airways before the alveoli may be represented by extruding the circle of the final generation out to an equivalent volume.

After generating the horn replica, each generation may be grouped according to biological regions or algorithmic determination. The hydraulic diameter may be then calculated for each grouping as a volume-weighted average or equally weighted across the group generations. From a database of lattice geometries, printable configurations with equivalent hydraulic diameter may be identified as candidates for airway approximation. Preferred candidates may have a smaller dimensionless radius, defined as the ratio of the strut radius to the cell length.

Following selection of a lattice unit cell and configuration, the diameters of the horn replica may be scaled by the reciprocal square root of unit cell porosity to match interstitial velocity through the porous media to the average airway velocity. The horn volume at each generation may be scaled in the radial direction as necessary, to maintain post-latticing void volume within an approximate range of the target generational quantity. The pre-alveolar extruded volume may be scaled axially to maintain void volume equal to the remaining generational volume. The latticed lobe approximation regions may consist of multiple distinct lattice forms, up to the total number of generations represented in the approximation. The latticed regions may be produced via additive manufacturing and may be dimensionally tuned to achieve uniform or non-uniform element thickness.

To create the manufacturable shell of the horn replica, a 0.5-5 mm wall thickness may be added to a surface created by smooth transition (loft) between the diameters of the generation equivalents and the extrusion of the final generation. As necessary, the diameter of the first approximated generation may be adjusted to match or exceed the circumscribed outlet airway diameter. A mate fitting and corresponding mounting posts may be added to fit the lobe shell to the airway outlet at the tracheobronchial tree. At the base of the shell, a groove for an o-ring may be cut into the shell. The lobe approximation shells may be produced by additive manufacturing or molding.

After the lobe approximation shell and lattice regions are designed, an enclosed form of equal volume of terminal alveoli may be modelled at the end of the lobe approximation unit, taking the form of a hemisphere, bellows, or frustum (referred to as the alveolar volume). The shape negative may be designed in a multi-part assembly to create a mold of the enclosed form, from which the volume may be cast from a flexible material, such as silicone, with Shore hardness in the range of Shore 00 30 to Shore A 30. After casting, the base of the alveolar volume may be fitted with an attachment point for actuation by the motion control system.

Finally, for each lobe, the lobe approximation shell, latticed regions, and alveolar volume may be assembled as a single unit and attached to the model replica.

In an alternative case, all lobe approximation units may be identical.

In an alternative case, the alveolar volume form may be created directly by an additive manufacturing process.

### Breathing

Once the lung replica has been produced, an arbitrary breathing profile may be prescribed through coordinated motion of the five lobes or 18 segments, each of which may be guided by a series of motion control devices (stepper motors or pneumatic actuators) in mechanical communication with the terminal lobe units. The lobe units may move synchronously or asynchronously, depending on the experimentally desired physiological alignment and availability or generation of relevant waveforms. Breath actuation may vary in duration, number of cycles, hold time, peak flow rate, and direction (inhalation vs exhalation). Breathing profile may be measured at the individual lobe level or through the mouth, using existing devices based on thermal mass principle or indirect velocity measurement.

Control of individual lobe units and modular components may be directed via a single microcontroller, one stepper motor per terminal unit, and one stepper motor driver per terminal unit. As one example, the respective components may be an Arduino MEGA 2560, StepperOnline 23HS30-3004S, and StepperOnline DM542T. The timing of motion may be directed by the delay between electrical pulses sent to the motor, which may be used to alter the shape of the breathing waveform between a sinusoidal form (representative of resting, tidal breathing) or a piecewise, biased sinusoidal waveform (representative of forced inspiration or expiration). The airflow output may be dependent upon both the motor speed and alveolar volume form. Exact prescription of the motor timing may be performed by first recording a constant-timing breath profile for a given alveolar volume configuration, then adjusting motor timing to achieve a target volume displacement in a given unit of time, minimizing the difference between the intended breathing profile and the measured result. Arbitrary breathing profiles may be generated by function-based or spline-based descriptions of motor delays, which may be uploaded to the Arduino.

In an alternative case, each modular component may be controlled by its own respective microcontroller.

### B. Adjustment

The lung replica enables adjustment of generation of upper airway truncation, use of mouth or nose and idealization. This may accommodate personalization and/or sex/age differences for grouped population assessments, and use as much patient-specific data as available.

The lung replica enables adjustment of lobe approximation features including equivalent diameter, “generation length”, truncation of terminal generation, length of pre-alveolar region, alveolar volume and shape of alveolar volume.

The lung replica enables adjustment of lattice unit cell selection, cell length, feature dimension (strut radius or surface thickness).

For lobe approximation and lattice parameter selection, optimal parameters may replicate the volume of each generation and lobe, the surface area of each generation, and for each generation, the average velocity (approximated by interstitial velocity in porous media). Successful configurations may also replicate total and regional deposition efficiency, aligned with rigorous published studies such as Heyder, Stahlhofen, and Conway (Heyder J, Gebhart J, Rudolf G, Schiller CF, Stahlhofen W. Deposition of particles in the human respiratory tract in the size range 0.005-15 μm. Journal of Aerosol Science 1986; 17:811-25. DOI: 10.1016/0021-8502 (86) 90035-2; Stahlhofen W, Rudolf G, James AC. Intercomparison of Experimental Regional Aerosol Deposition Data. Journal of Aerosol Medicine 1989; 2:285-308. DOI: 10.1089/jam. 1989.2.285; Conway J, Fleming J, Majoral C, Katz I, Perchet D, Peebles C, et al. Controlled, parametric, individualized, 2-D and 3-D imaging measurements of aerosol deposition in the respiratory tract of healthy human subjects for model validation. Journal of Aerosol Science 2012; 52:1-17. DOI: 10.1016/j.jaerosci.2012.04.006).

The lung replica enables adjustment of breathing profile to align with various breathing maneuvers and levels of effort, and use as much patient-specific data as available.

The lung replica enables adjustment of the materials the model is created of, and may be used from 3D printed resins.

The lung replica enables adjustment of the environmental conditions.

### C. Measurements

Dimensional characterization of the as-printed lattice inserts may verify agreement within 30% of the as-designed parameters. CT scans of lattice parts may be used to confirm parted-part accuracy against the as-designed part.

Airflow may be measured at each lobe, as well as the total airflow at the mouth of the lung replica. This may be important for the function of the replica and verifying disease modeling, mimicking spirometry measurements performed in human patients.

Aerosol deposition may be measured at each generation, which may be the critical function/output of the replica. This may be performed by a number of complementary methods on both the fully assembled replica model and deconstructed lattices using appropriate techniques for the given aerosol. For 3D imaging of spatial aerosol deposition on the lattices, magnetic resonance imaging (MRI) or CT, using appropriate contrast aerosols (i.e. iron oxide, gold contrast, respectively) may be performed. Confocal microscopy, leveraging fluorescent tags, may also be employed for high spatial resolution of select lattice regions. For chemical analysis, quantification on washes of deconstructed lattices via appropriate analytical techniques (i.e. inductively coupled-mass spectrometry [ICP-MS], high-performance liquid chromatography [HPLC], mass spectrometry [MS], or fluorescence). With each lattice region easily segmented, deposition maps of total aerosols collected in each region may be readily established through appropriate solvent washes of the lattices and surrounding lobe wall, as well as the upper airway throat. For fluorescent particles, detection of fluorescence in the rinsed collections may readily be converted to a particle concentration and total deposition amount per washed region.

Exhaled fraction may be measured by collecting aerosols not deposited on the lung replica and quantified with the same methods as above. This may be done by inserting a chamber over the mouth during the exhalation maneuver.

In an alternative case, the exhaled fraction may be determined by closing the mass balance from deposited dose and the original dosage amount.

### D. Applications

Replica can measure deposition of therapeutics and environmental aerosols The end point of the replica may be used to measure regional aerosol deposition at the lobe to generation level. Measuring where this deposition occurs may be directly linked to health impacts of the inhaled agent. This may lead to a wide variety of benefits.

Use of the replica may be applied to developing new inhalers and therapeutics by understanding where they deposit.

Use of this replica may be used to measure environmental aerosol (i.e. wildfire smoke, workplace hazards) by placing the replica in various exposure environments. By assessing regional deposition of these inhaled agents, inputs to exposure and toxicology assessments may be performed.

Replica can promote the development of new inhaler devices, attachments to existing aerosol generating device, new inhalable medicines, and exhalation monitoring devices

Existing inhaler devices may include metered dose inhalers (MDI), dry powder inhalers (DPI), and nebulizers. An improved preclinical lung replica may lead to enhanced assessment of new inhaler devices prior to clinical trials, allowing for assessment of aerosol deposition under imposed breathing profiles. Use of this replica model may allow for realistic behavior of both the inhaler (DPI/MDI/nebulizer) dynamics and respiratory physics in a single device to establish realistic measures of currently unobtainable scenarios. These may include novel devices timed to breathing maneuvers.

Similar to the development of new inhaler devices, improved methods of preclinical assessment may improve predictability of where aerosols may deposit under certain breathing conditions, improving the understanding and design of new inhaler attachments (i.e., spacers). These may include novel regional targeting devices and mouthpieces.

Improved methods of preclinical assessment may improve throughput of formulation development. This may include new aerosol formulations ranging from prophylactic and therapeutic agents (e.g., small molecule, biologics and particulate) and may include both new molecules and/or new formulations.

Improved methods of measuring deposition and exhaled fraction may lead to development of new devices to measure exhaled aerosols. These may be used for health analyses: biomarkers, metabolites, breathalyzers, etc.

In all scenarios, the replica may be used to measure aerosol deposition under a range of patient simulated breathing maneuvers. From the resultant data, iteration of the new device design may be performed to target a desirable range of deposition profiles (i.e., uniform coverage, increased localization to a certain lobe/generation etc.). Varied breathing profiles may provide a sensitivity analysis for the overall target performance.

Replica may promote the validation of generic inhaled medicines (equivalence)

Specific to generic inhalable drugs, this replica provides opportunity to measure bioequivalence (BE) of orally inhaled nasal drug products (OINDPs). The replica may establish in a single platform the whole dose deposition equivalence (DE) under dynamic and patient-specific conditions. This may lead to increased understanding linking formulation parameters to airway deposition through use of standardized breathing profiles and highly controlled in vitro experimentation that may advance the state-of-the-art understanding of respiratory aerosol dynamics. Fundamental advances in these areas may ultimately lead to enhanced assessment of generic OINDPs. For OINDP generics, this may directly translate to long-term reductions in clinical trial BE validation and a unique in vitro alternative to preclinical large animal OINDP deposition studies, supporting targets set by the European Union and U.S. Environmental Protection Agency (targeting complete elimination by 2035). This may further facilitate a pipeline towards establishing BE of many OINDPs.

Similar to development of new aerosols, the replica may be used to measure aerosol deposition of the BE OINDP under a range of patient simulated breathing maneuvers. From the resultant data, iteration of the BE OINDP (either formulation or device) may be performed to target a desirable range. Population Bioequivalence (PBE) Analysis as recommended by the FDA may readily be applied and varied breathing profiles may provide a sensitivity analysis for the overall target performance.

Replica may provide evidence to establish exposure/deposition guidelines for new and existing aerosol agents (cigarettes, e-cigarettes, workplace chemicals, radiological)

Knowing where hazardous aerosols may deposit in the lung will allow for toxicological assessments. Paired with other biological assessments, deposition data provided by the lung replica may provide important input information to establish exposure criteria. Deposition data from the lung replica may be input into exposure models and paired with biological information to establish guidelines.

Replica may provide preclinical deposition data of otherwise inaccessible patient populations.

In certain patient populations, aerosol deposition may be altered based on anatomical or disease-specific airway features. Sometimes, trial deposition data is not feasible to obtain (i.e. premature babies, critically ill patients, rare structural airway diseases, pediatric patients, elderly). The lung replica may establish preclinical deposition patterns for patient populations that deposition data does not exist to provide an important set of information in treating these patients (through design of new devices, formulations etc.). In this case, the replica may be designed to incorporate patient data and used to measure aerosol deposition under a range of patient simulated breathing maneuvers. From the resultant data, evaluation of existing and/or personalized treatment paradigms may be pursued.

### Replica May Provide Data Needed in Clinical Bridging Studies

In clinical development, a bridging study may be required as a supplemental study to extrapolate existing clinical data to a new parameter space. This replica may provide a critical piece of deposition information needed to reduce the uncertainty of how the drug may perform under the new scenario (patient demographic, breathing change, formulation/device difference etc.). Deposition in the lung replica may be performed under the prior and changed parameter space and predictions to downstream outputs may be generated.

### Replica May be Used to Establish Clinical Guidelines for New and Generating Aerosol Therapies

Knowing where an advantageous aerosol is deposit in the lung may allow for assessments of how effective that treatment may be in varied patient populations. Paired with other biological assessments, deposition data provided by the lung replica may provide important input information to establish preclinical therapeutic criteria that may predict clinical outcomes. The replica may be used to measure aerosol deposition under a range of patient simulated breathing maneuvers for the new aerosol. From the resultant data, iteration of the new device design may be performed to target a desirable range of preclinical deposition profiles (i.e., uniform coverage, increased localization to a certain lobe/generation etc.). Varied breathing profiles may provide a sensitivity analysis for the overall target performance. Once preclinical data has been established, the correlation to clinical outcomes (following appropriate trials) may be evaluated, allowing for refinement of the lung replica.

### Replica May be Used to Customize Existing or New Aerosol Treatments

The modularity of the replica approach allows for distinct patient features to be readily incorporated, leading to personalized inhaler devices and formulations to be tested. This could include new devices or formulations for groups of individuals with similar patient parameters (breathing, volume, structure), anticipating changes to deposition profiles with disease progression and adjusting formulation/device/dosing procedure to improve therapeutic benefit, or point-of-care personalized deposition predictions and adjusted inhaler prescription. In all cases, patient data may be integrated into the replica, deposition data obtained under appropriate breathing maneuvers, and iterations of the appropriate therapy performed on preclinical scenarios before translating to patient treatments.

Replica May Interface with Existing Physiological Based Pharmacokinetic (PBPK) Models and Lead to Improved Predictions

Existing PBPK models may readily accept the spatial deposition information generated by the lung replica as inputs to downstream compartment modeling. The higher resolution data (down to the generation level) and lead to further refinement and improved PBPK model predictions of airway retention, tissue dosage, and blood profiles. Deposition data generated from the replica under a prescribed breathing maneuver may be lumped into the existing inputs for the given PBPK model and run as normal. Increased granularity to the PBPK may be designed to match those of our replica for higher resolution predictions.

### E. “Personal Touches”

a. Elastic Properties

Elastic properties of the deformable parts must allow for both tension and compression. Silicones have been ideal for this application.

b. Special Conditions

Environmental control may be achieved by placing the replica in an enclosed chamber around the apparatus to regulate temperature (T), relative humidity (RH), and particulate matter levels. Humidity control within the replica may be generated from one or more of the following: 1) attached humidified air delivery systems at the distal ends of the lobe units, 2) attached humidified air delivery systems through ports incorporated into the anatomy-derived upper airway components, or 3) attached humidified air delivery systems incorporated into idealized regions up upper airway components. Temperature control of the replica using conductive elements in contact with the upper airway replica or lobe units, may be separate from environmental control measures within the enclosure. Conditions to mimic the lung may include internal temperature of 100% relative humidity at 37° C.

### C. Alternative

Non-periodic porous media may be used in place of lattices, as long as repeatable structures could be achieved.

All documents, books, manuals, papers, patents, published patent applications, guides, abstracts, and/or other references cited herein are incorporated by reference in their entirety. Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

1. A lung replica, comprising:
(a) an upper airway compartment comprising an upper airway space;
(b) one or more lobe compartments, wherein each of the one or more lobe compartments comprises two or more generation regions, a compressible terminal unit, and lattices in the two or more generation regions, and each of the one or more lobe compartments comprises a lobar airway space connected with the upper airway space; and
(c) one or more modular components, wherein the one or more modular components actuate the one or more lobe compartments to compress or expand, whereby an exhaled airflow is generated when the one or more lobe compartments are compressed, and an inhaled airflow is generated when the one or more lobe compartments are expanded.

2. The lung replica of claim 1, wherein the lung replica comprises 1-18 lobe compartments.

3. The lung replica of claim 1, wherein each of the lobe compartments comprises 2-23 generation regions.

4. The lung replica of claim 1, wherein the lung replica has a total fully expanded lung airway volume of 0.1-7 L.

5. The lung replica of claim 1, wherein the lung replica has a total fully compressed lung airway volume of 0.01-3 L.

6. The lung replica of claim 1, wherein the upper airway compartment comprises a wall on the outer surface of the upper airway compartment, wherein the wall has a thickness of 0.1-10 mm.

7. The lung replica of claim 1, wherein the upper airway compartment comprises a proximal compartment and a distal compartment, wherein the proximal compartment comprises a first opening, and the distal compartment is connected with the one or more lobe compartments.

8. The lung replica of claim 7, wherein the proximal compartment further comprises a second opening.

9. The lung replica of claim 1, wherein the lung replica has a total fully expanded lobar airway volume of 0.1-7 L.

10. The lung replica of claim 1, wherein the lung replica has a total fully compressed lobar airway volume of 0.01-3 L.

11. The lung replica of claim 1, wherein lune replica has a total lobe internal surface area of 1-100 m².

12. The lung replica of claim 1, wherein each of the one or more lobe compartments comprises a shell on the outer surface of each of the one or more lobe compartments, wherein the shell has a thickness of 0.1-10 mm.

13. The lung replica of claim 1, wherein the shell has a Shore hardness in the range from Shore 00 30 to Shore D 90.

14. The lung replica of claim 1, wherein the terminal unit has a fully expanded alveolar airway volume of 0.1-7 L.

15. The lung replica of claim 1, wherein the terminal unit is in the form of a sealed hemisphere, bellows, or frustum.

16. The lung replica of claim 1, wherein each of the two or more generation regions has a unique property selected from the group consisting of porosity, specific surface area and hydraulic diameter.

17. The lung replica of claim 1, wherein the lattices have a porosity (ε) of 0.1-1.

18-26. (canceled)

27. A method for exchanging airflow in the lung replica of claim 1, comprising:
(a) compressing the one or more lobe compartments, whereby an exhaled airflow is generated; and
(b) expanding the one or more lobe compartments, whereby an inhaled airflow is generated, whereby the lung replica exchanges airflow.

28-39. (canceled)

40. A method for preparing a lobe compartment, wherein the lobe compartment comprises a lobe airway space and a generation region based on an analogous airway generation in an analogous lobe, comprising
(a) forming the lobar airway space in the lobe compartment with a series of connected circles having gradually changing diameters along the length of the lobe compartment;
(b) adding a lattice into a circle in the connected circles to form a generation region, wherein the generation region has a cross sectional area based on a cumulative cross sectional area of the analogous airway generation, scaled in inverse proportion to porosity of the lattice, and a depth based on a length of the analogous airway generation; and
(c) forming a terminal unit in the lobe compartment, whereby the lobe compartment is prepared.

41-42. (canceled)

43. A method for generating a lung replica, comprising
(a) providing an upper airway compartment, wherein the upper airway compartment comprises an upper airway space;
(b) providing one or more lobe compartments prepared according the method of claim 40;
(c) connecting the upper airway space with the lobar airway space in each of the one or more lobe compartments; and
(d) providing one or more modular components, wherein the one or more modular components actuate the one or more lobe compartments to compress or expand, whereby an exhaled airflow is generated when the one or more lobe compartments are compressed, and an inhaled airflow is generated when the one or more lobe compartments are expanded.
